(19) United States
(12) Patent Application Publication
(10) Pub. No.: US 2007/0009819 A1
(43) Pub. Date: Jan. 11, 2007

(54) Novel compound, and electrophotographic photoreceptor and electrophotographic apparatus made by using the same

(76) Inventors: Nobuyuki Sekine, Hachioji, Tokyo (JP); Masami Kuroda, Zushi, Kanagawa (JP); Kenichi Ohkura, Tokyo (JP); Yoshihiro Ueno, Matsumoto, Nagano (JP); Motohiro Takeshima, Nagano (JP)

Correspondence Address: RABIN & Berdo, PC, 1101 14TH STREET, NW, SUITE 500, WASHINGTON, DC, 20005, US

(21) Appl. No.: 10/562,859

(22) PCT Filed: Jul. 1, 2004
(86) PCT No.: PCT/JP04/09317

§ 371 Date: Jun. 5, 2006

(30) Foreign Application Priority Data
Jul. 2, 2003 (JP) 2003-190022
Nov. 26, 2003 (JP) 2003-395769
Dec. 8, 2003 (JP) 2003-408433

(51) Int. Cl. G03G 15/02; C09B 29/32; C07D 409/02

(52) U.S. Cl. 430/58.15; 534/739; 534/740

(57) ABSTRACT

A compound useful for a positive charge type electrophotographic photoreceptor with high sensitivity for copiers and printers by using such a novel organic material as a charge transport substance in a photosensitive layer and an electrophotographic apparatus using the same. The compound has a structure represented by the following general formula (I). 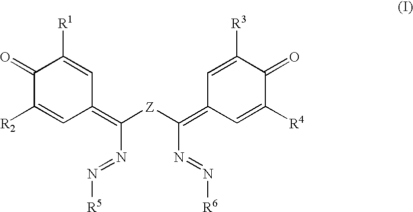
(In the general formula (I), R¹, R², R³, and R⁴ each is an alkyl group or the like; R⁵ and R⁶ each-is an aryl group or the like; and Z represents a structure having the following general formula (F-A), (F-B) or (F-C). 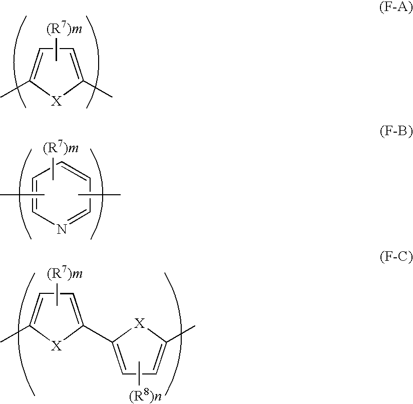
(In the formulae, R⁷ and R⁸ each is an optionally substituted alkyl group; m and n each represents an integer of from 0 to 2; and X is a sulfur atom or an oxygen atom.).

### TECHNICAL FIELD

The present invention relates to a novel compound and in detail, it relates to a novel compound which is useful as a charge transport substance in an electrophotographic photoreceptor (hereinafter also referred to simply as “photoreceptor”) and the like. Also, the invention relates to an electrophotographic photoreceptor and an electrophotographic apparatus and in detail, it relates to an electrophotographic photoreceptor comprising an electrically conductive substrate having thereon a photosensitive layer containing an organic material, which is used in printers or copiers of the electrophotographic system and the like, and to an electrophotographic apparatus.

### BACKGROUND ART

Hitherto, as a photosensitive layer of an electrophotographic photoreceptor, an inorganic photoconductive substance such as selenium and selenium alloys or a dispersions of an inorganic photoconductive substance such as zinc oxide and cadmium sulfide in a resin binding agent has been employed. In recent years, studies regarding electrophotographic photoreceptors using an organic photoconductive substance have advanced, some being put into practical use while improving sensitivity and durability.

Furthermore, the photoreceptor is required to hold a surface charge in a dark place, a receive light to generate a charge, and similarly receive light to transport a charge. There are so-called single-layer-type photoreceptors in which a single layer has these functions at the same time. There are also so-called layered-type photoreceptors in which layers which have been functionally separated into a layer for principally contributing to the generation of a charge and a layer for contributing to holding of a surface charge in a dark place and charge transportation at the time of receiving light are laminated.

For the image formation by electrophotography using such a photoreceptor, for example, the Carlson system has been applied. The image formation according to this system is carried out by electrically charging a photoreceptor in a dark place by corona discharge, forming an electrostatic latent image of letters, a picture, etc. of an original on the surface of the electrically charged photoreceptor, developing the formed electrostatic latent image with a toner, and fixing the developed toner image onto a support such as paper. After the toner image is transferred to the support, the photoreceptor is subjected to neutralization, removal of a residual toner, optical neutralization, and the like and then provided for reuse.

In comparison with inorganic photoreceptors, organic photoreceptors that are put into practical use are advantageous in view of their flexibility, film forming properties, low cost, safety, etc. Additionally, due to the diversity of materials, further improvements in sensitivity, durability, and the like are being advanced.

Most organic photoreceptors are layered-type organic photoreceptors in which the functions are separated into a charge generation layer and a charge transport layer. In general, in the layered type organic photoreceptor, a charge generation layer containing a charge generation substance such as pigments and dyes, and a charge transport layer containing a charge transport substance such as hydrazine and triphenylamine are formed in this order on an electrically conductive support. Due to the properties of the charge transport substance, which is electron-donating, this layered-type organic photoreceptor is of a hole-transfer type and, when the surface of the photoreceptor is negatively charged, has sensitivity. Now, in a negative charge type photoreceptor, the corona discharge to be applied at the time of charging becomes unstable as compared with a positive charge type photoreceptor. Also, since ozone and nitrogen oxides are generated, they are adsorbed on the surface of the photoreceptor so that physical or chemical deterioration is liable to occur, and this has a negative effect upon the environment. From these viewpoints, in comparison with the negative charge type photoreceptor, the positive charge type photoreceptor having a high degree of freedom in use, is broad in its range of applications and advantageous as the photoreceptor.

Then, in order to use a photoreceptor of the positive-charge type, there is proposed a method for simultaneously dispersing a charge generation substance and a charge transport substance in a resin binder to use the photoreceptor in the form of a single photosensitive layer, and some have been put into practical use. However, the single-layer-type photoreceptor is not sufficiently sensitive that it may be applied in a high-speed machine, and further improvements are necessary in repeatability and the like.

Furthermore, in order to take a layered structure whose functions are separated for the purpose of achieving high sensitivity, a method for laminating a charge generation layer on a charge transport layer to form a positive charge type photoreceptor may be considered. However, according to this system, since the charge generation layer is formed on the surface, there is a problem of inadequate stability during repeated use, etc. caused by corona discharge, light irradiation, mechanical abrasion, etc. In this case, it is also proposed to further provide a protective layer on the charge generation layer. However, though the problem of mechanical abrasion is reduced, a problem of, for example, bringing a reduction of electrical characteristics such as sensitivity is not overcome yet.

In addition, there is also proposed a method for laminating a charge transport layer for transporting electrons onto a charge generation layer to form a photoreceptor.

As an electron transport substance, for example, 2,4,7-trinitro-9-fluorenone and the like is known. However, this substance is problematical with regard to safety because it is carcinogenic. Besides, in the below-listed Patent Documents 1 to 4 and so on, cyano compounds and quinine-based compounds are proposed. However, ones having electron transport ability sufficient for practical use have not been obtained yet.

In addition, for example, in the below-listed Patent Documents 5 to 15 and Non-Patent Documents 1 to 4, and so on, a number of electron transport substances and electrophotographic photoreceptors using the same are proposed and described, and get attention. Furthermore, for example, as described in the below-listed Patent Documents 16 to 20, photoreceptors using a combination of a hole transport substance and an electron transport substance in a single layer type photosensitive layer get attention for the reason that they have high sensitivity, and some have been put into practical use.

On the other hand, the present inventors have proposed a variety of photoreceptors containing a substance having excellent electron transport properties (for example, those described in the below-listed Patent Documents 21 to 25).

Patent Document 1: JP-A-1-206349

Patent Document 2: JP-A-6-59483

Patent Document 3: JP-A-9-190002

Patent Document 4: JP-A-9-190003

Patent Document 5: JP-A4-360148

Patent Document 6: JP-A-3-290666

Patent Document 7: JP-A-5-92936

Patent Document 8: JP-A-9-151157

Patent Document 9: JP-A-5-279582

Patent Document 10: JP-A-7-179775

Patent Document 11: JP-A-10-73937

Patent Document 12: JP-A-4-338760

Patent Document 13: JP-A-1-230054

Patent Document 14: JP-A-8-278643

Patent Document 15: JP-A-2001-222122

Patent Document 16: JP-A-5-150481

Patent Document 17: JP-A-6-130688

Patent Document 18: JP-A-9-281728

Patent Document 19: JP-A-9-281729

Patent Document 20: JP-A-10-239874

Patent Document 21: JP-A-2000-75520

Patent Document 22: JP-A-2000-199979

Patent Document 23: JP-A-2000-143607

Patent Document 24: JP-A-2001-142239

Patent Document 25: JP-A-2001-215742

Non-Patent Document 1: The Journal of Imaging Society of Japan, Vol. 30, pp. 266-273 (1991)

Non-Patent Document 2: Preprint of Pan-Pacific Imaging Conference/Japan Hardcopy '98, July 15-17, 1998, JA HALL, Tokyo, Japan, pp. 207-210

Non-Patent Document 3: Collected Papers of Japan Hardcopy '97, July 9, 10 and 11, 1997, JA HALL (Otemachi, Tokyo), pp. 21-24

Non-Patent Document 4: Collected Papers of Japan Hardcopy '92, July 6, 7 and 8, 1992, JA HALL (Otemachi, Tokyo), pp. 173-176

As described above, a variety of investigations with respect to a charge transport substance having electron transport properties have hitherto been made. However, in recent years, according to a requirement for photoreceptors with high sensitivity, there has been a demand for a photoreceptor with high performance using a new charge transport substance having better electron transport properties.

### OBJECT AND SUMMARY OF THE INVENTION

Therefore, an object of the invention is to provide a compound having excellent electron transport ability which is useful for applications in an electrophotographic photoreceptor. It is a further object of the invention to provide a positive charge type electrophotographic photoreceptor with high sensitivity for copiers and printers by using such a novel organic material as a charge transport substance in a photosensitive layer and an electrophotographic apparatus using the same.

In order to achieve the foregoing objects, the present inventors made extensive and intensive investigations regarding a variety of organic materials. As a result, it has been found that by using, as a charge transport substance, a specific compound having electron transport properties as represented by the following general formula (I), it is possible to obtain a photoreceptor with high sensitivity which can be used in a positive charge process, leading to accomplishment of the invention.

Specifically, in order to solve the foregoing problems, the invention is concerned with a novel compound which is characterized by having a structure represented by the following general formula (I): 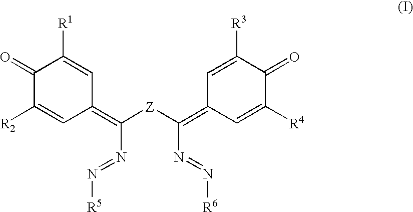
(in the general formula (I), R¹, R², R³, and R⁴ may be the same or different and each is a hydrogen atom, an optionally substituted alkyl group having from 1 to 12 carbon atoms, or an optionally substituted aryl group; R⁵ and R⁶ may be the same or different and each is an optionally substituted aryl group or an optionally substituted heterocyclic group; and Z represents a structure having the following general formula (F-A), (F-B) or (F-C): 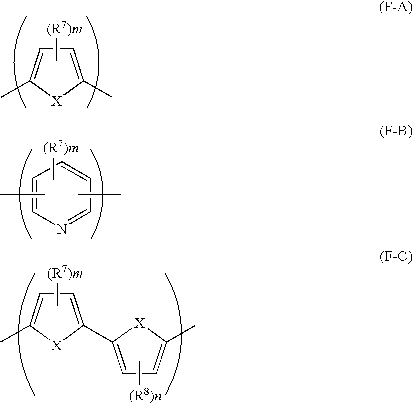
(in the formulae, R⁷ and R⁸ may be the same or different and each represents an optionally substituted alkyl group having from 1 to 12 carbon atoms; m and n each represents an integer of from 0 to 2; X represents a sulfur atom or an oxygen atom; and each substituent is a halogen atom, a nitro group, an alkyl group, an aryl group, a heterocyclic group, a halogenated alkyl group, or an alkoxy group, and the substituents may be taken together to form a ring).

Furthermore, in order to solve the foregoing problems, the invention is concerned with an electrophotographic photoreceptor comprising an electrically conductive substrate having thereon a photosensitive layer containing a charge generation substance and a charge transport substance, which is characterized in that the subject photosensitive layer contains at least one kind of the foregoing novel compound (I).

In addition, the invention is concerned with an electrophotographic apparatus which is characterized by being provided with the foregoing electrophotographic photoreceptor of the invention and performing a charge process by a positive charge process.

According to the invention, it is possible to obtain a compound having excellent electron transport properties, and by applying this compound to an electronic device using an organic compound, such as electrophotographic photoreceptors, it becomes possible to enhance electrical characteristics and the like. Furthermore, according to the invention, since this compound is used as a charge transport substance in a photosensitive layer as provided on an electrically conductive substrate, it has become possible to obtain a photoreceptor with high sensitivity and excellent electrical characteristics in positive charge. Moreover, since it is possible to choose, as a charge generation substance, a substance which is suitably adaptive to the type of exposure light source, by using a phthalocyanine compound, a squarylium compound, a bisazo compound, etc., it is possible to obtain a photoreceptor which can be used in semiconductor laser printers or copiers. In addition, if desired, it is possible to enhance durability by providing a covering layer on the surface.

### BRIEF DESCRIPTION OF THE DRAWINGS

FIG. 1 is a conceptual cross-sectional view to show a single layer type electrophotographic photoreceptor.

FIG. 2 is a conceptual cross-sectional view to show a layered type electrophotographic photoreceptor.

FIG. 3 is an IR spectrum of a compound represented by the structural formula (I-A-3).

FIG. 4 is a ¹H-NMR spectrum of a compound represented by the structural formula (I-A-3).

FIG. 5 is an IR spectrum of a compound represented by the structural formula (I-A-5).

FIG. 6 is a ¹H-NMR spectrum of a compound represented by the structural formula (I-A-5).

FIG. 7 is an IR spectrum of a compound represented by the structural formula (I-B-4).

FIG. 8 is a ¹H-NMR spectrum of a compound represented by the structural formula (I-B-4).

FIG. 9 is an IR spectrum of a compound represented by the structural formula (I-B-7).

FIG. 10 is a ¹H-NMR spectrum of a compound represented by the structural formula (I-B-7).

FIG. 11 is an IR spectrum of a compound represented by the structural formula (I-B-12).

FIG. 12 is a ¹H-NMR spectrum of a compound represented by the structural formula (I-B-12).

FIG. 13 is an IR spectrum of a compound represented by the structural formula (I-C-1).

FIG. 14 is a ¹H-NMR spectrum of a compound represented by the structural formula (I-C-1).

### BEST MODE FOR CARRYING OUT THE INVENTION

Preferred embodiments of the invention will be hereunder described in detail.

First of all, a compound represented by the foregoing general formula (I) will be described.

Examples of the compound represented by the foregoing general formula (I) include compounds represented by the following general formula (I-A) in which Z in the general formula (I) is a structure represented by the foregoing general formula (F-A). 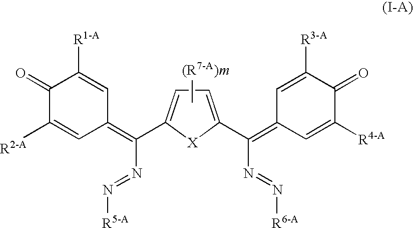
(In the formula (I-A), R^{1-A}, R^{2-A}, R^{3-A}, and R^{4-A} may be the same or different and each is a hydrogen atom, an optionally substituted alkyl group having from 1 to 12 carbon atoms, or an optionally substituted aryl group; R^{5-A} and R^{6-A} may be the same or different and each is an optionally substituted aryl group or an optionally substituted heterocyclic group; R^{7-A} is an optionally substituted alkyl group having from 1 to 12 carbon atoms; X represents a sulfur atom or an oxygen atom; m represents an integer of from 0 to 2; and each of the substituents is a halogen atom, a nitro group, an alkyl group having from 1 to 6 carbon atoms, an aryl group, a halogenated alkyl group having from 1 to 6 carbon atoms, or an alkoxy group having from 1 to 6 carbon atoms.). Specific examples of compounds represented by the foregoing general formula (I-A) will be given by the following structural formulae (I-A-1) to (I-A40), but it should not be construed that the invention is limited to these compounds. Incidentally, the symbol 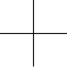
in the specific examples means a t-butyl group.

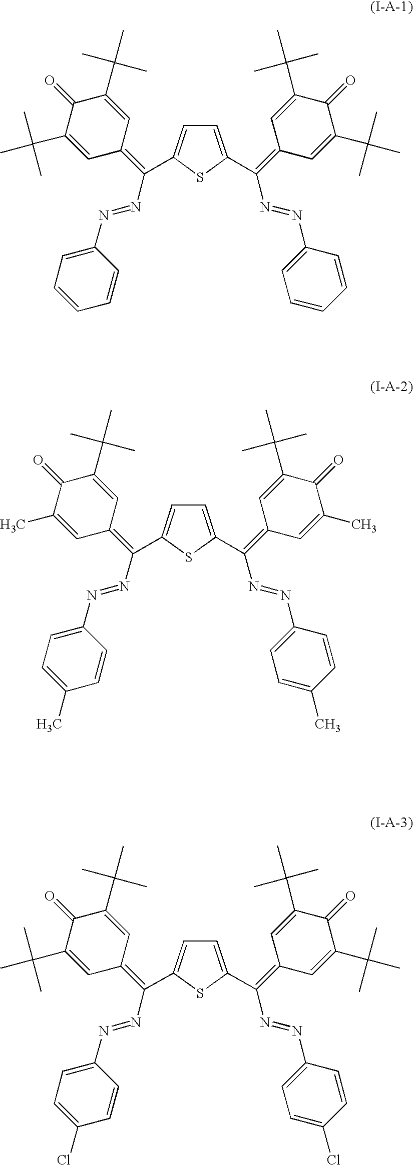

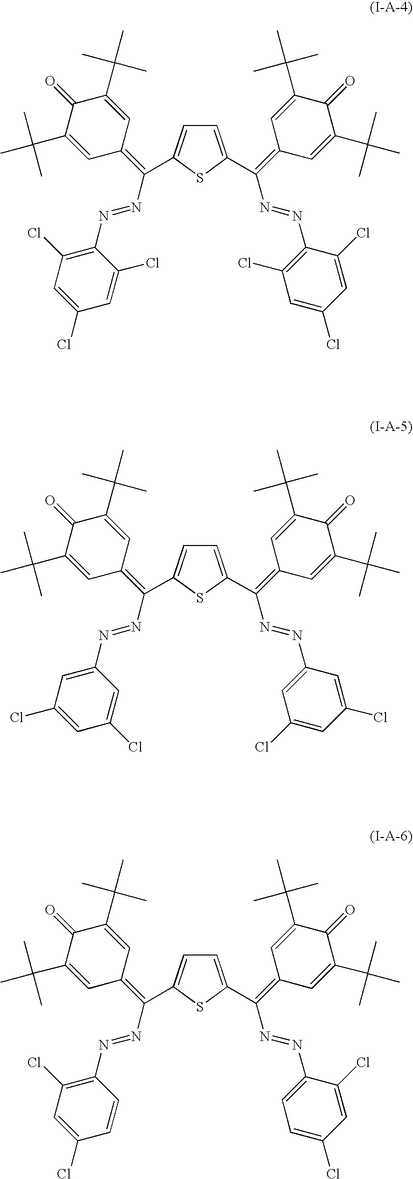

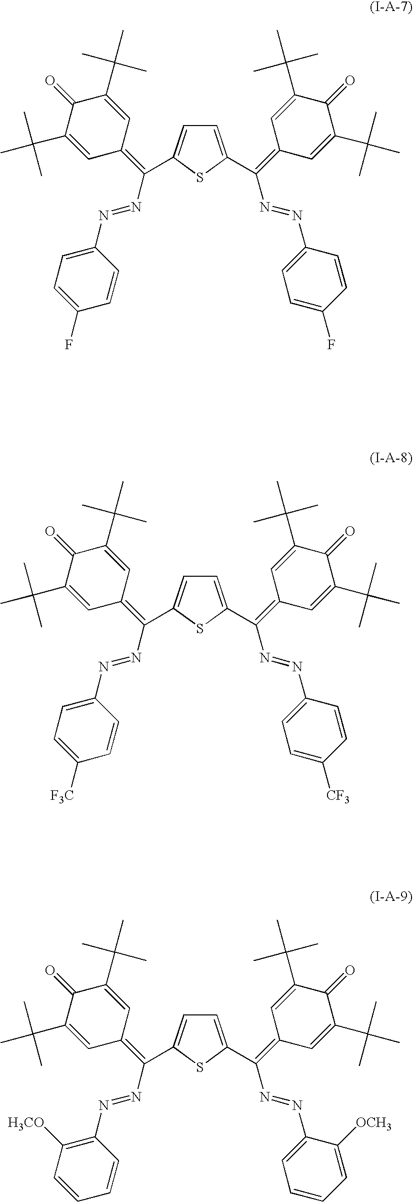

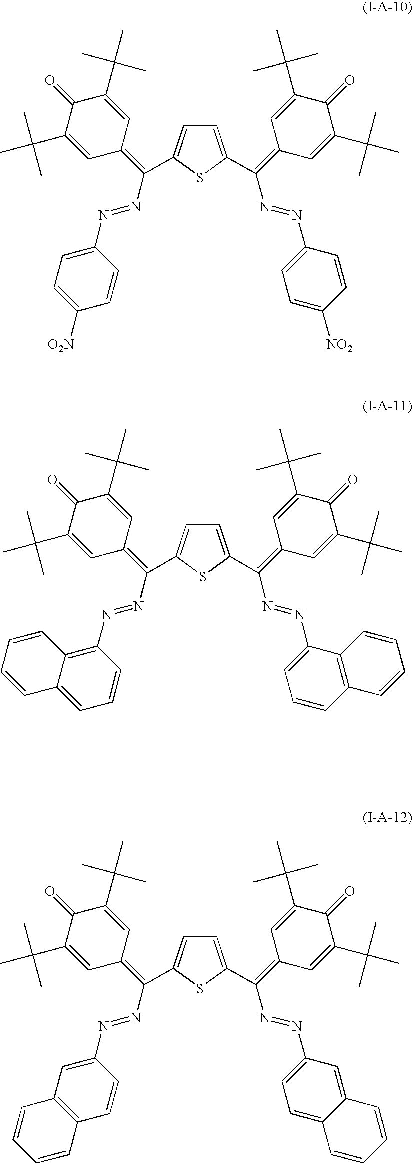

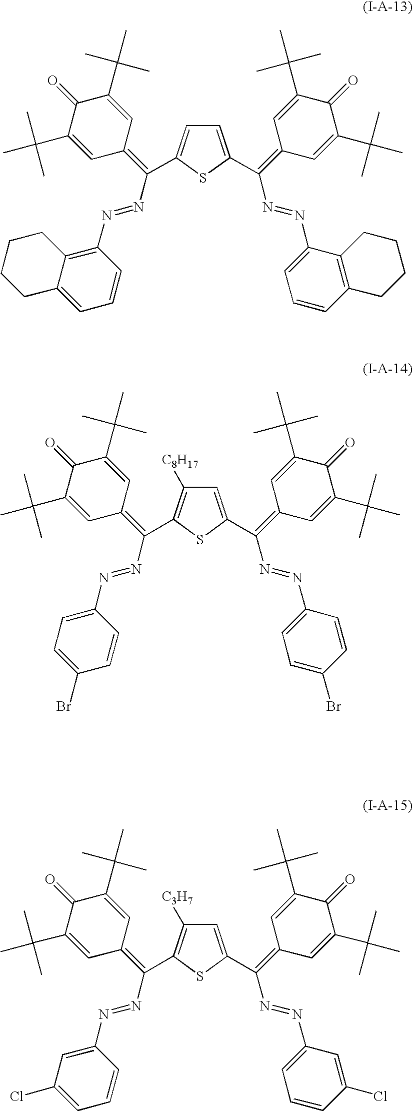

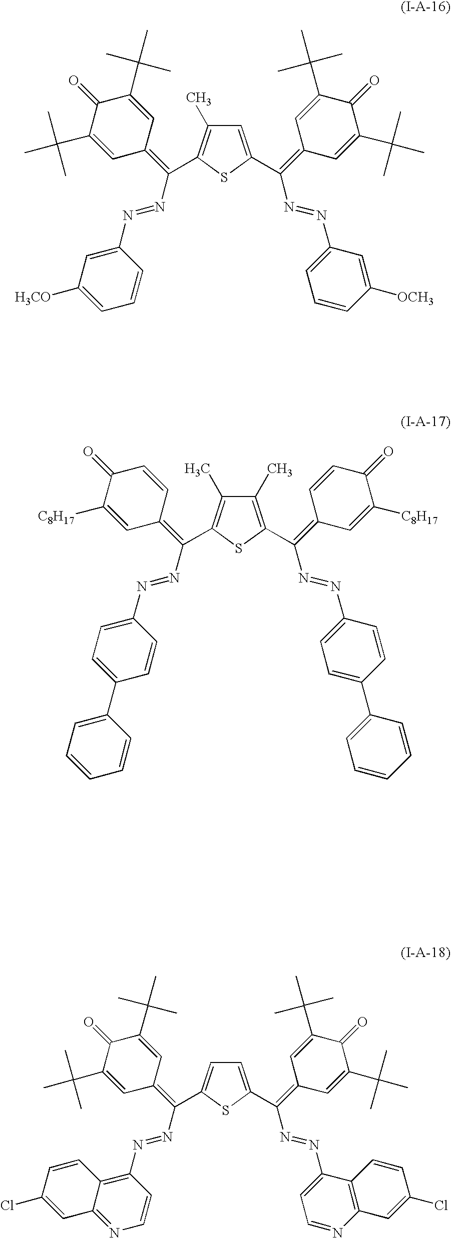

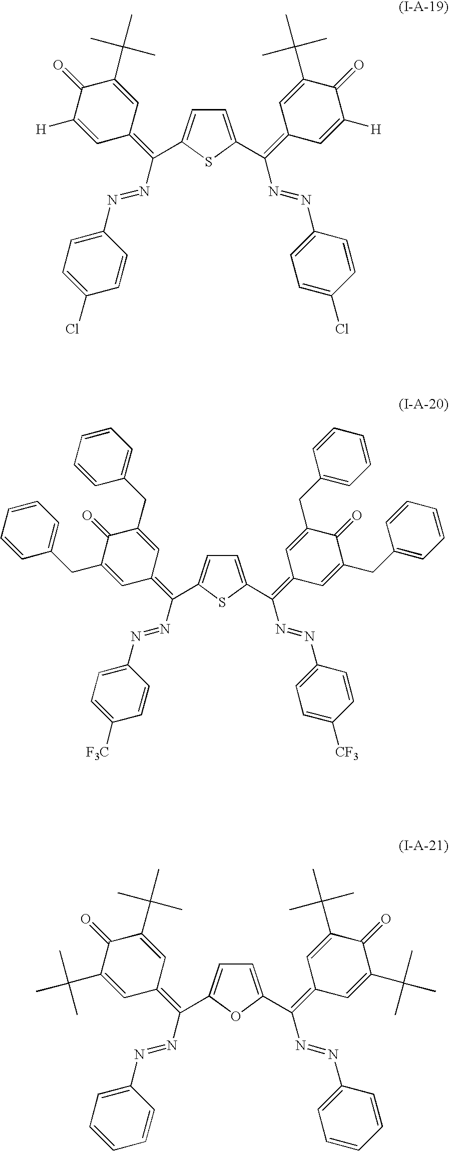

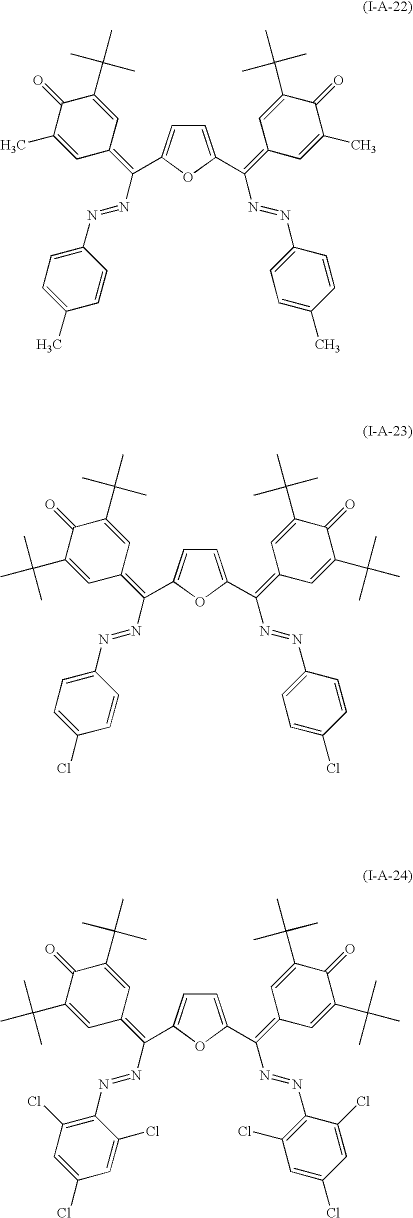

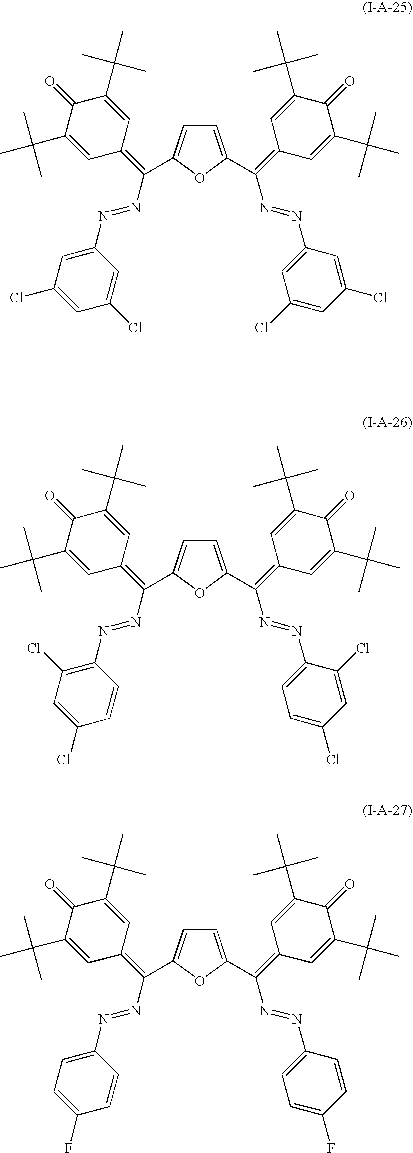

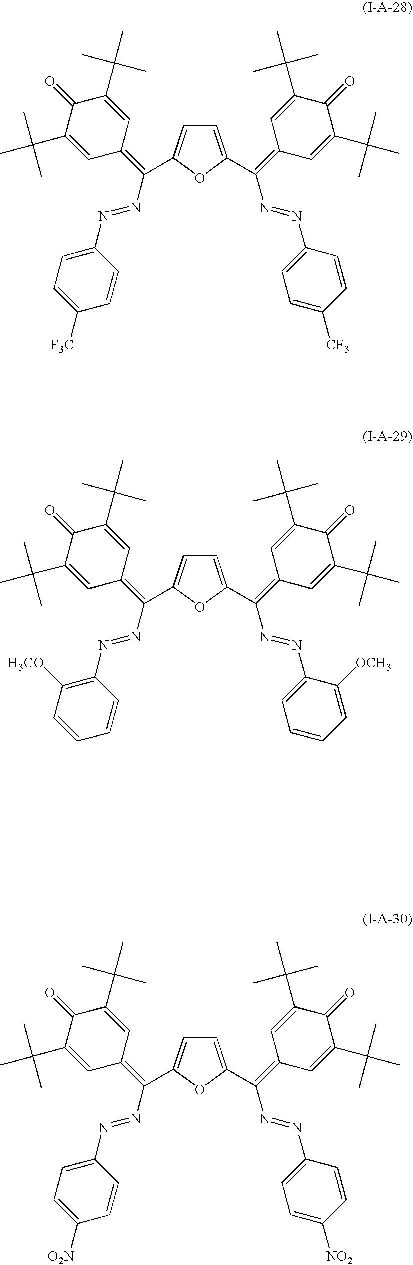

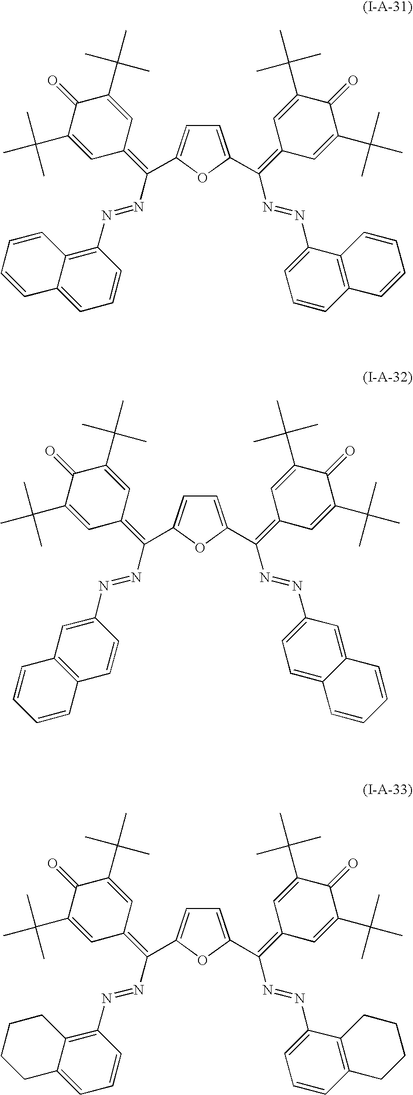

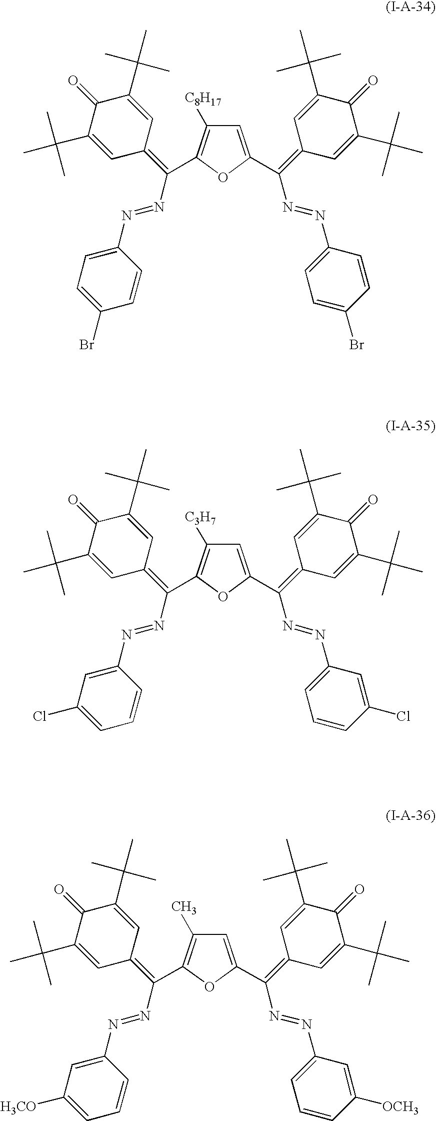

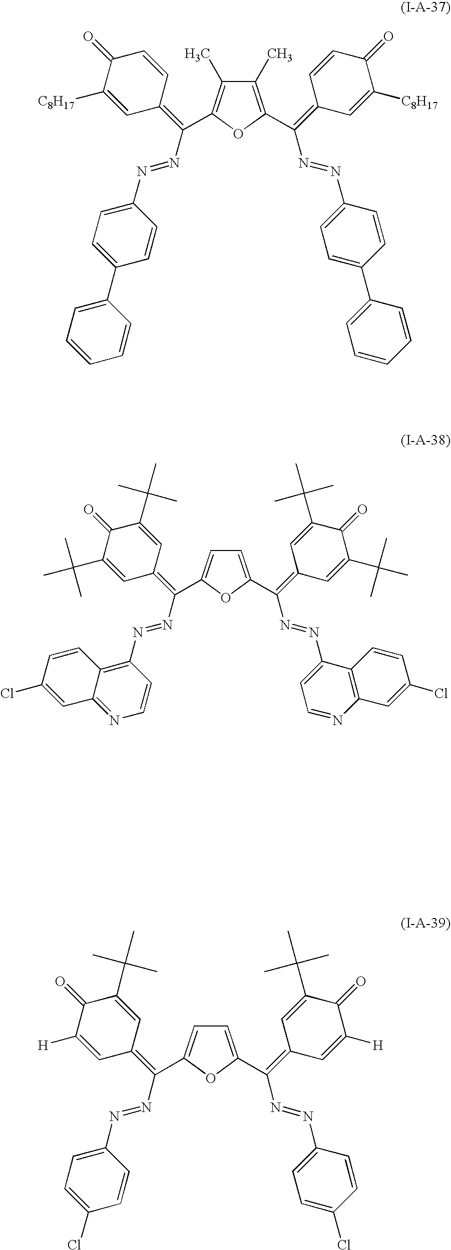

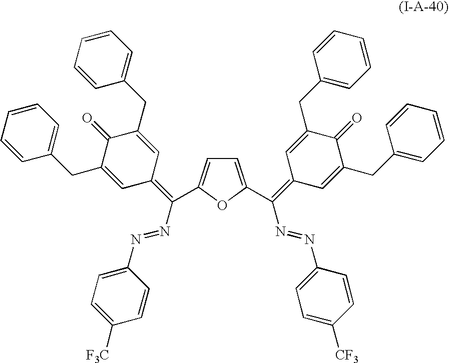

Furthermore, examples of the compound represented by the foregoing general formula (I) include compounds represented by the following general formula (I-B) in which Z in the general formula (I) is a structure represented by the foregoing general formula (F-B) (m=0). 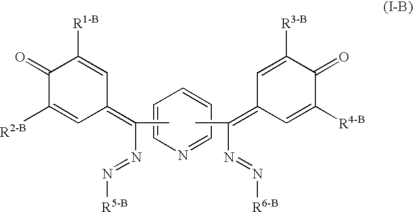
(In the formula (I-B), R^{1-B}, R^{2-B}, R^{3-B}, and R^{4-B} may be the same or different and each is a hydrogen atom or an optionally substituted alkyl group having from 1 to 12 carbon atoms; R^{5-B} and R^{6-B} may be the same or different and each is an optionally substituted aryl group or an optionally substituted heterocyclic group; and the substituents each is a halogen atom, an alkyl group, an alkoxy group, an aryl group, a heterocyclic group, a fluorinated alkyl group, or a nitro group, and the substituents may be taken together to form a ring.) Specific examples of compounds represented by the foregoing general formula (I-B) will be given by the following structural formulae (I-B-1) to (I-B-30), and specific examples of other compounds represented by the foregoing general formula (I) will be given by the following structural formulae (I-B-31) to (I-B-40). However, it should not be construed that the invention is limited to these compounds.

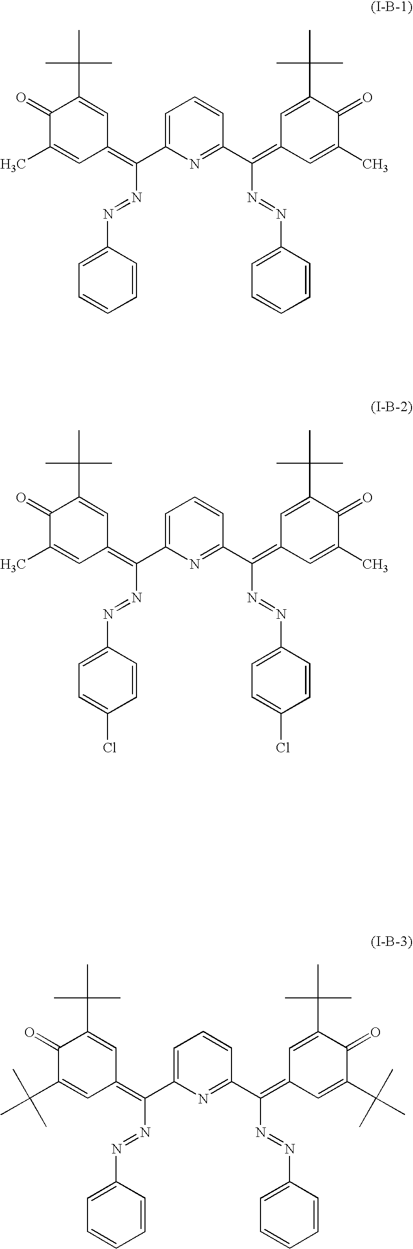

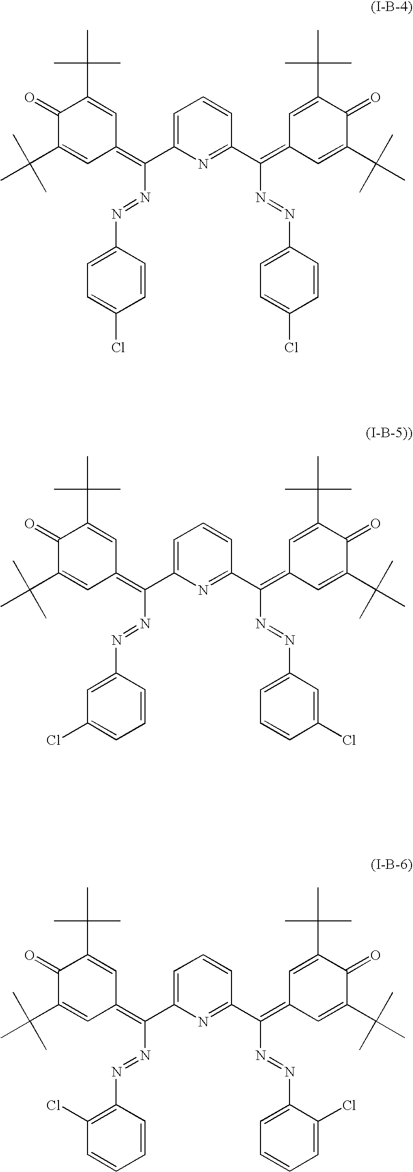

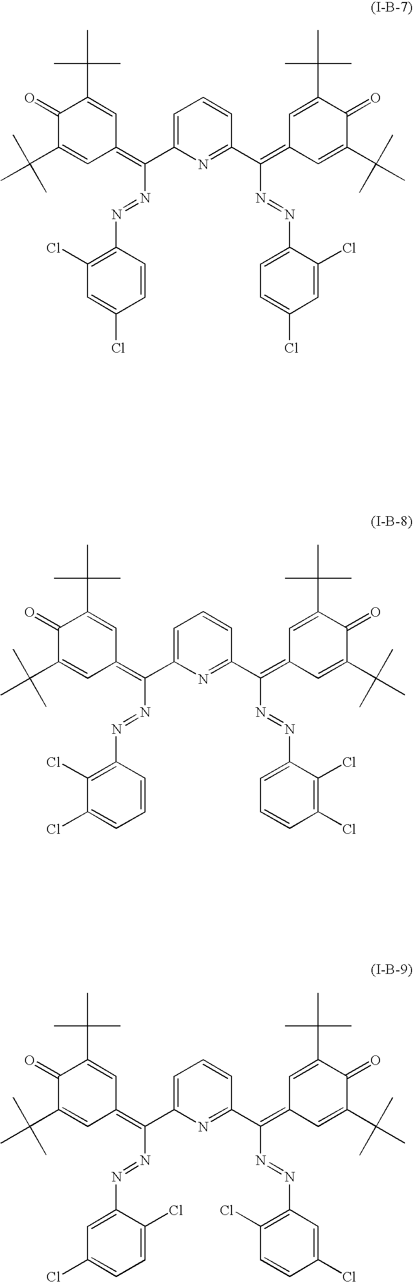

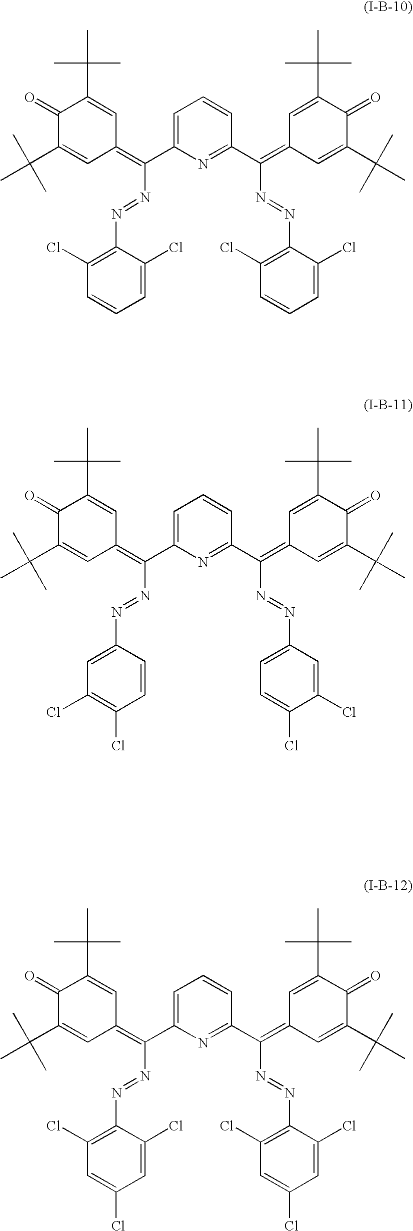

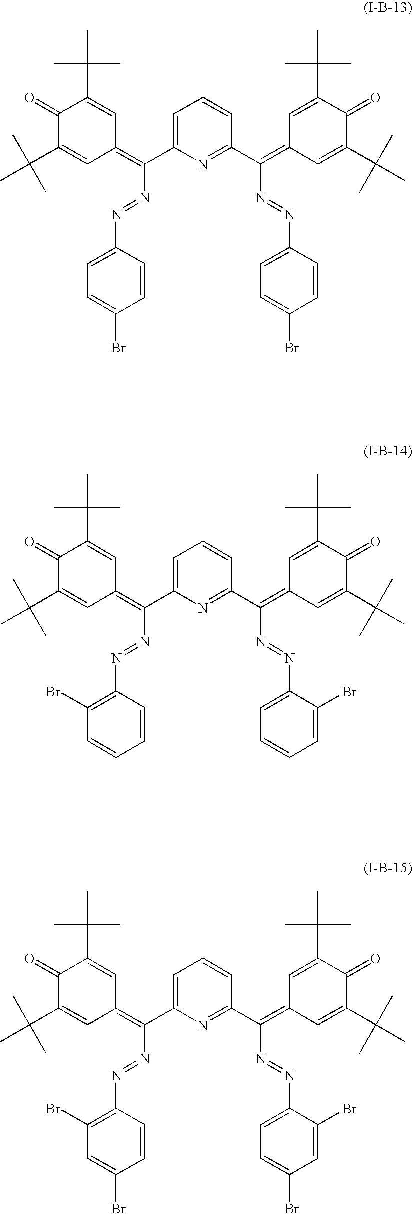

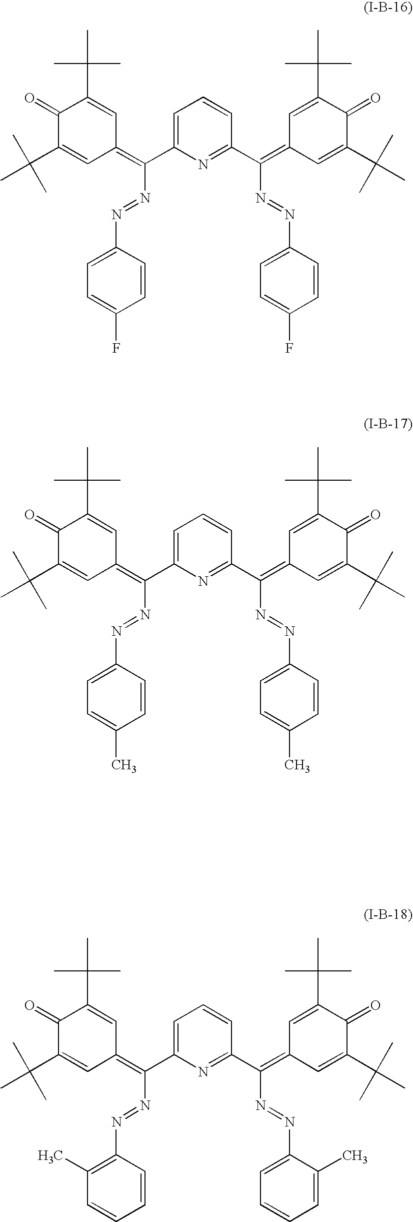

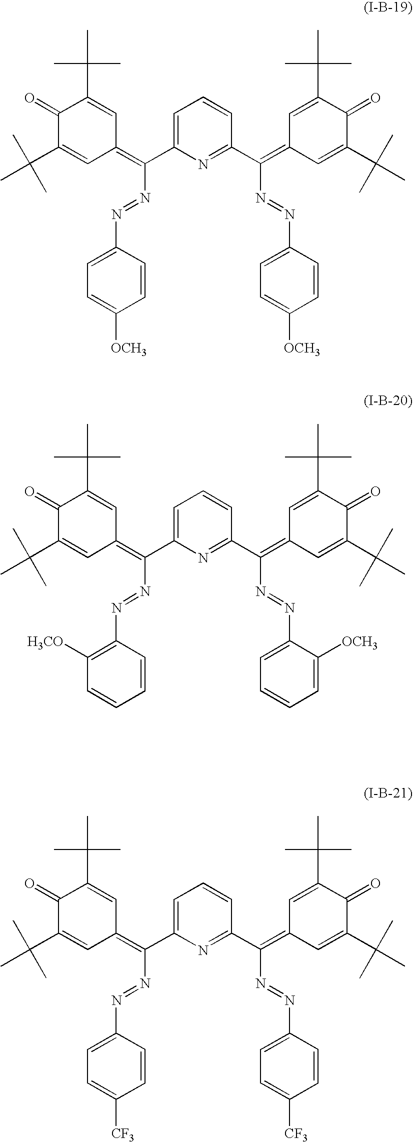

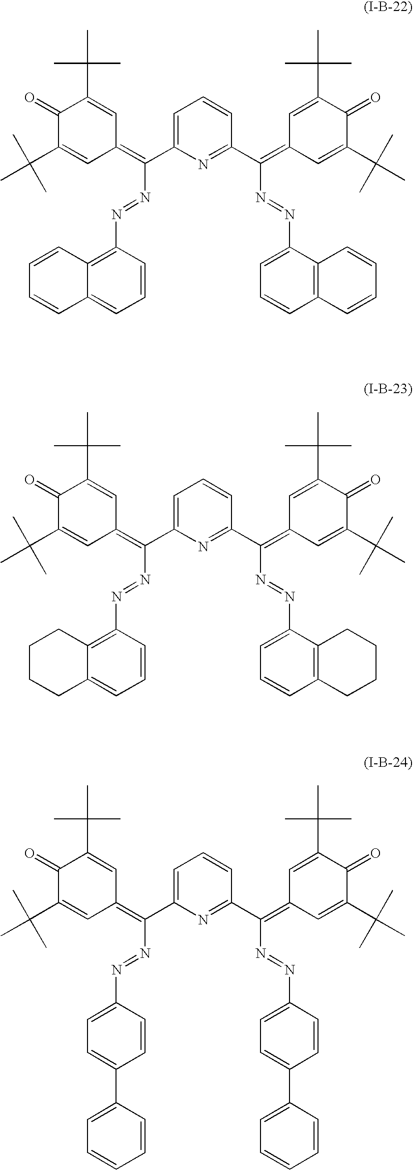

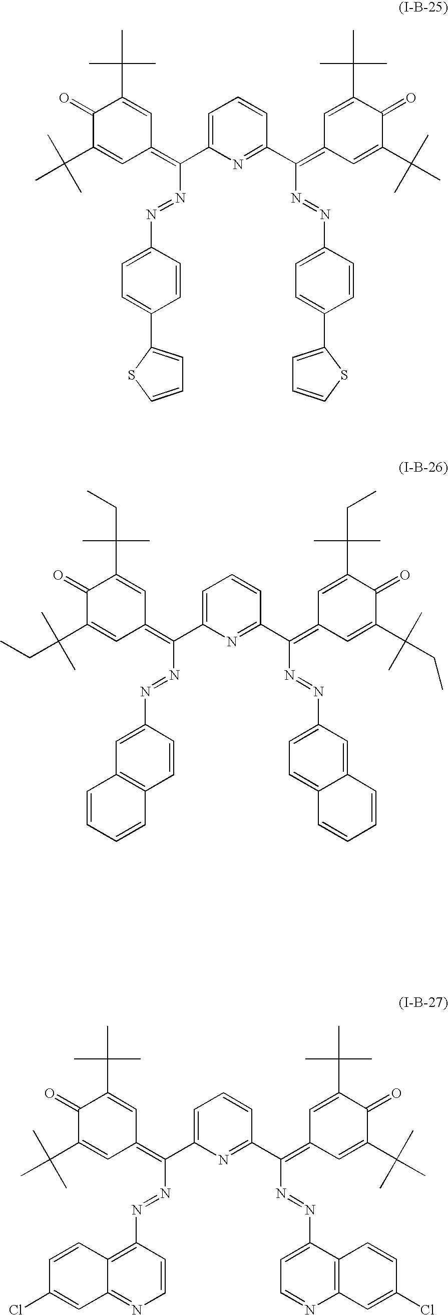

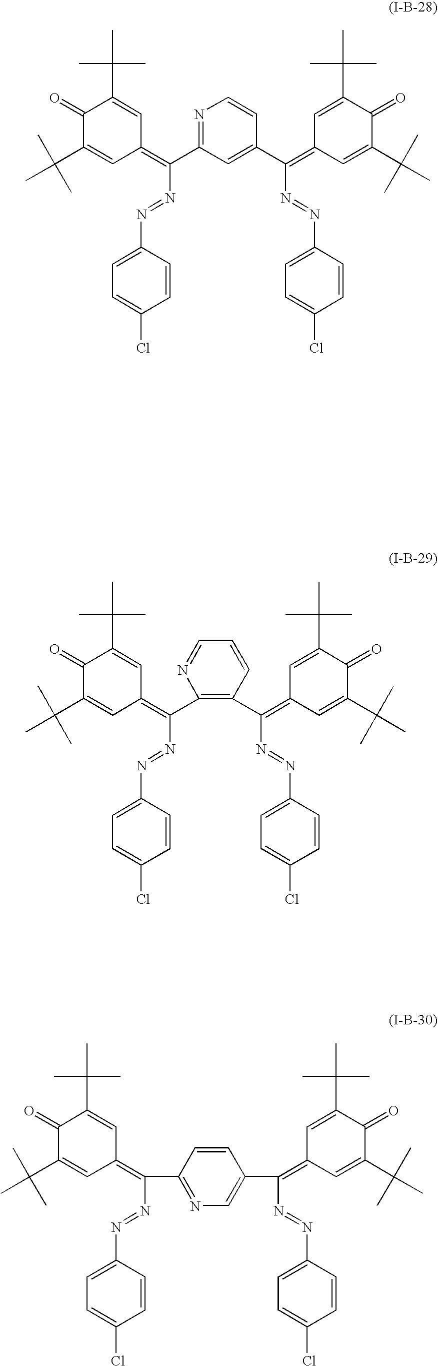

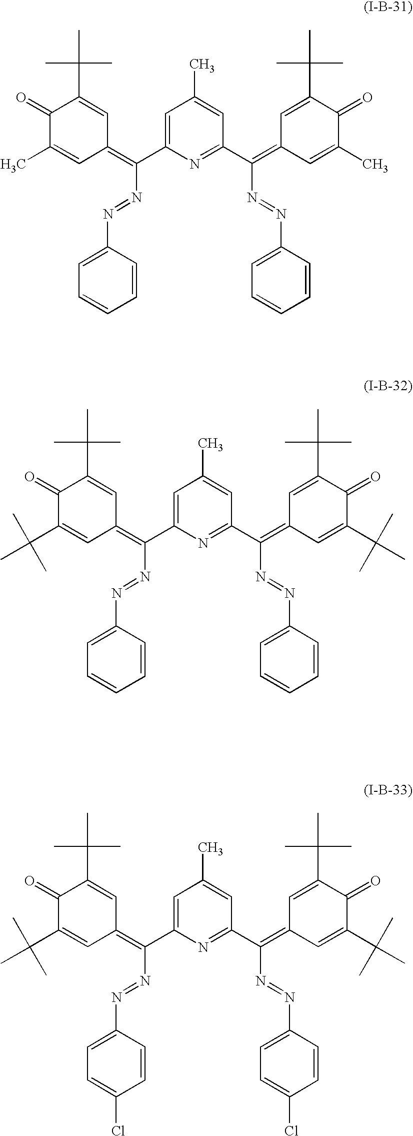

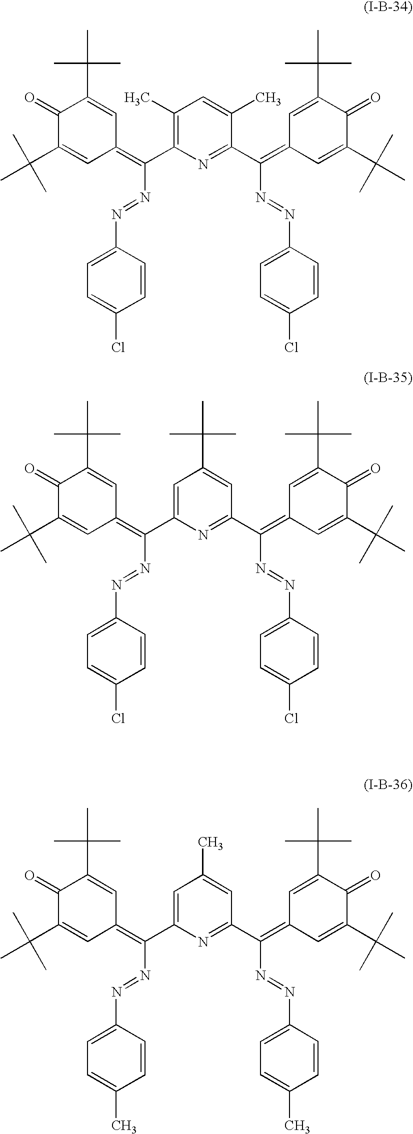

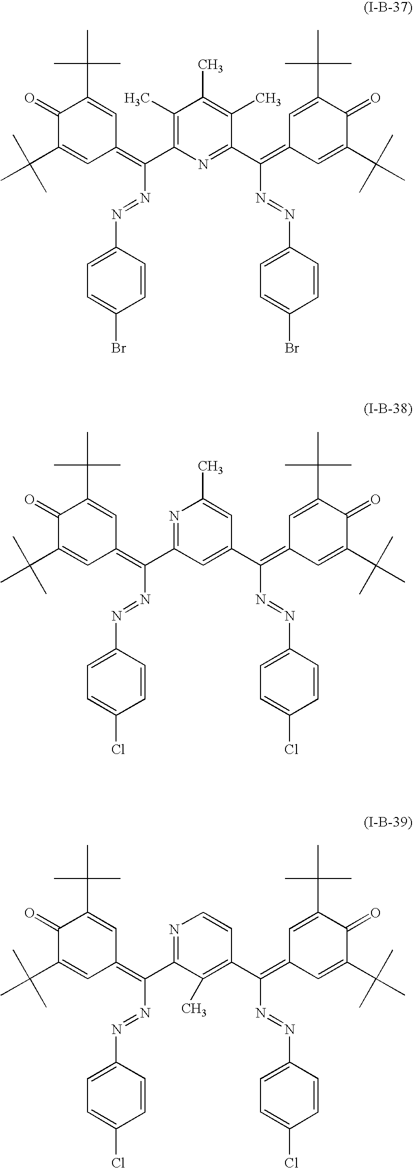

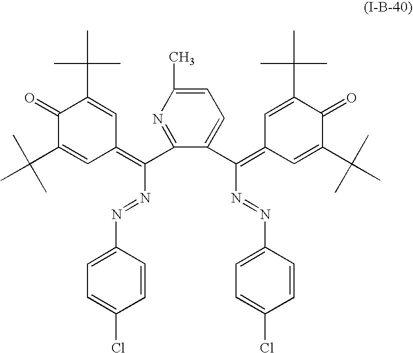

In addition, examples of the compound represented by the foregoing general formula (I) include compounds represented by the following general formula (I-C) in which Z in the general formula (I) is a structure represented by the foregoing general formula (F-C). 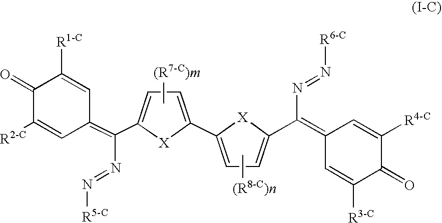
(In the formula (I-C), R^{1-C}, R^{2-C}, R^{3-C}, and R^{4-C} may be the same or different and each is a hydrogen atom, an optionally substituted alkyl group having from 1 to 6 carbon atoms, or an optionally substituted aryl group; R^{5-C} and R^{6-C} may be the same or different and each is an optionally substituted aryl group or a heterocyclic group; R^{7-C} and R^{8-C} each is a hydrogen atom or an optionally substituted alkyl group having from 1 to 10 carbon atoms; X represents a sulfur atom or an oxygen atom; m and n each represents an integer of from 1 to 2; and the substituents each represents a halogen atom such as chlorine and bromine, a nitro group, an alkyl group having from 1 to 6 carbon atoms such as a methyl group, a cycloalkyl group, an aryl group, a heterocyclic group, a halogenated alkyl group, or an alkoxy group having from 1 to 6 carbon atoms such as a methoxy group, and the substituents may be taken together to form a ring.) Specific examples of compounds represented by the foregoing general formula (I-C) will be given by the following structural formulae (I-C-1) to (I-C-19), but it should not be construed that the invention is limited to these compounds.

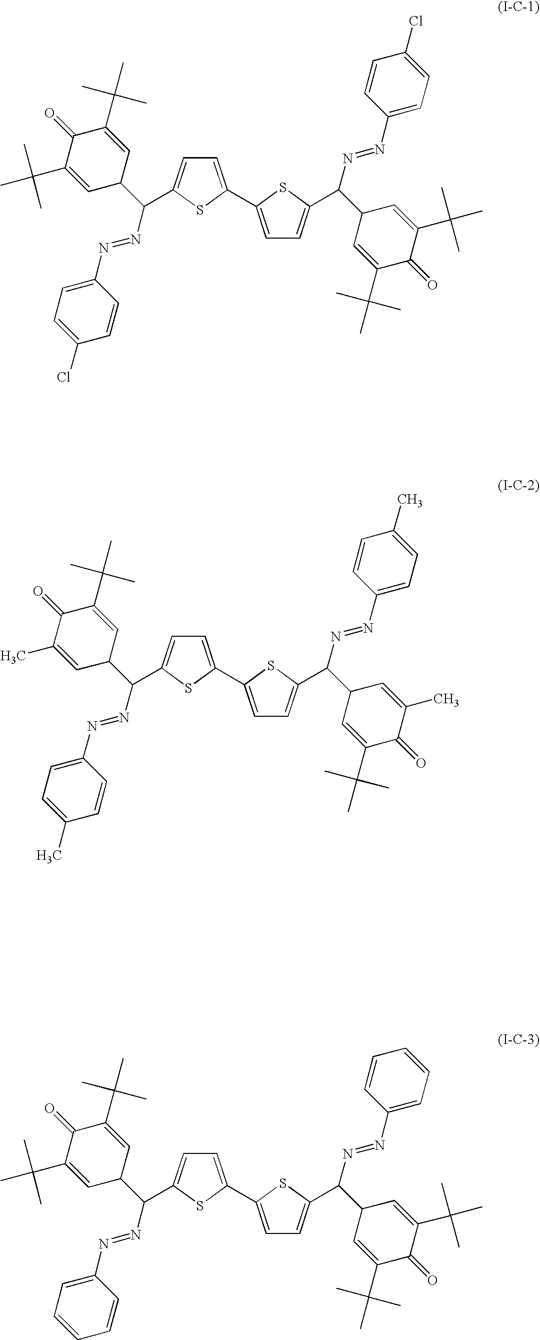

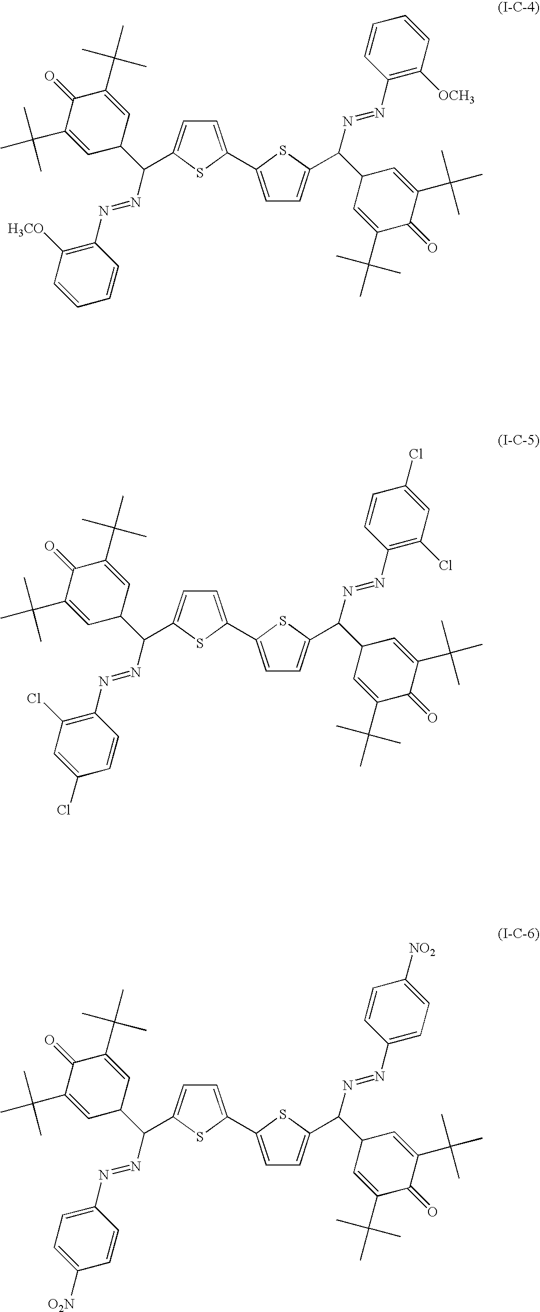

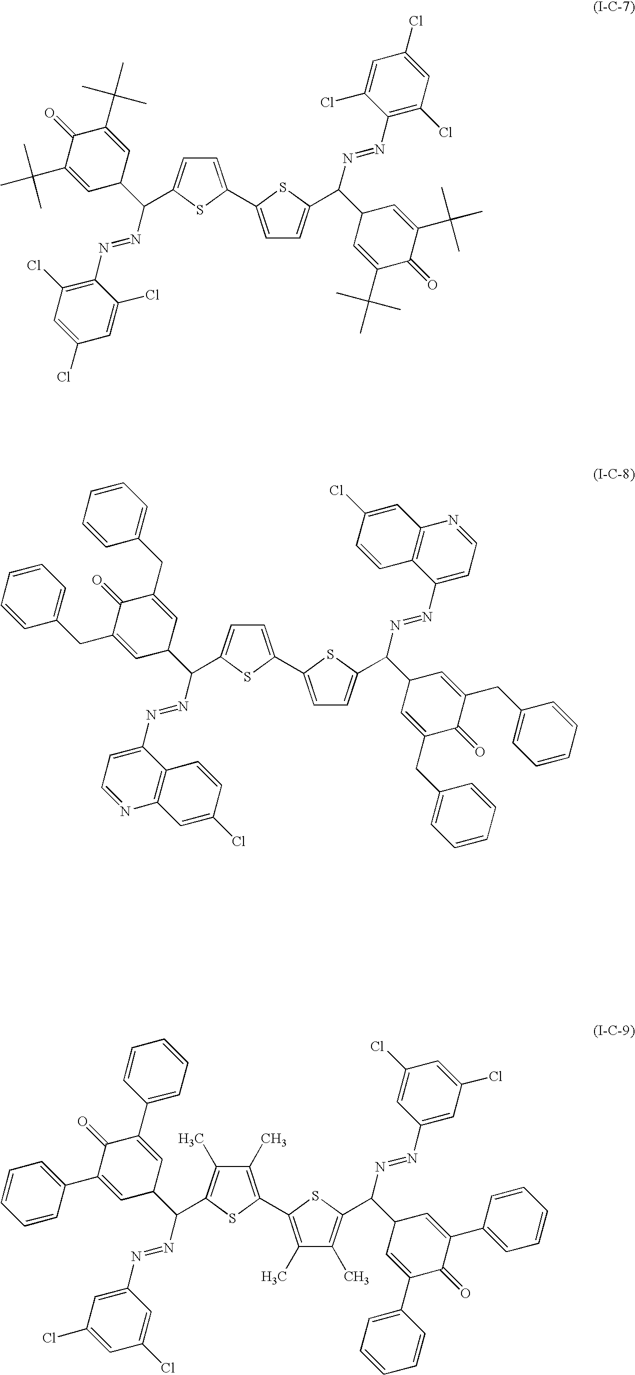

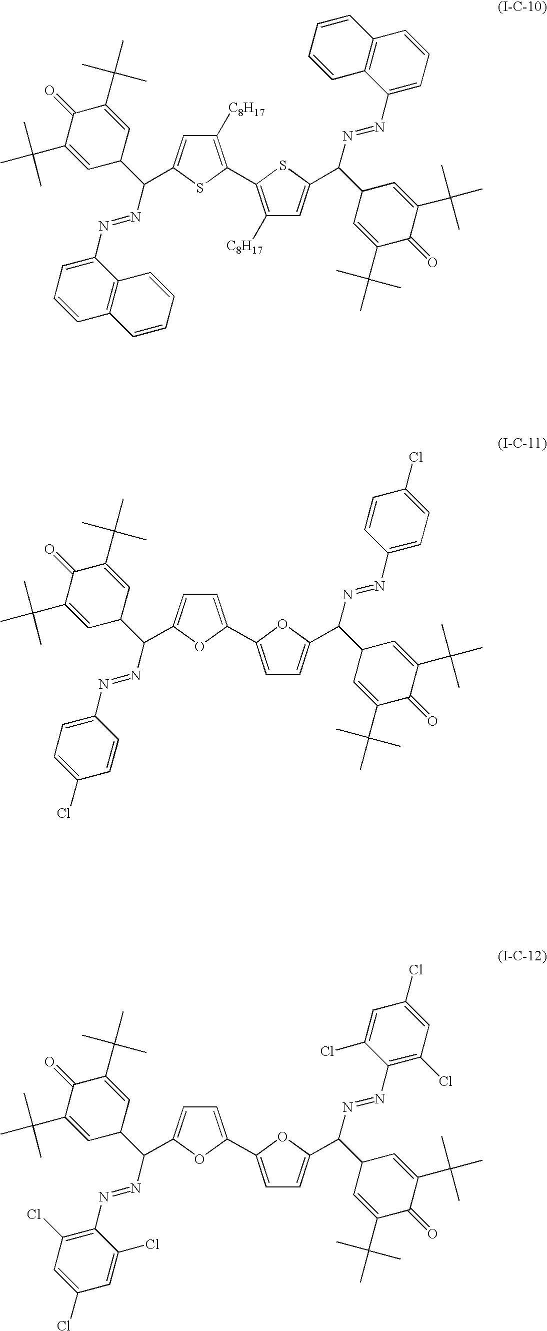

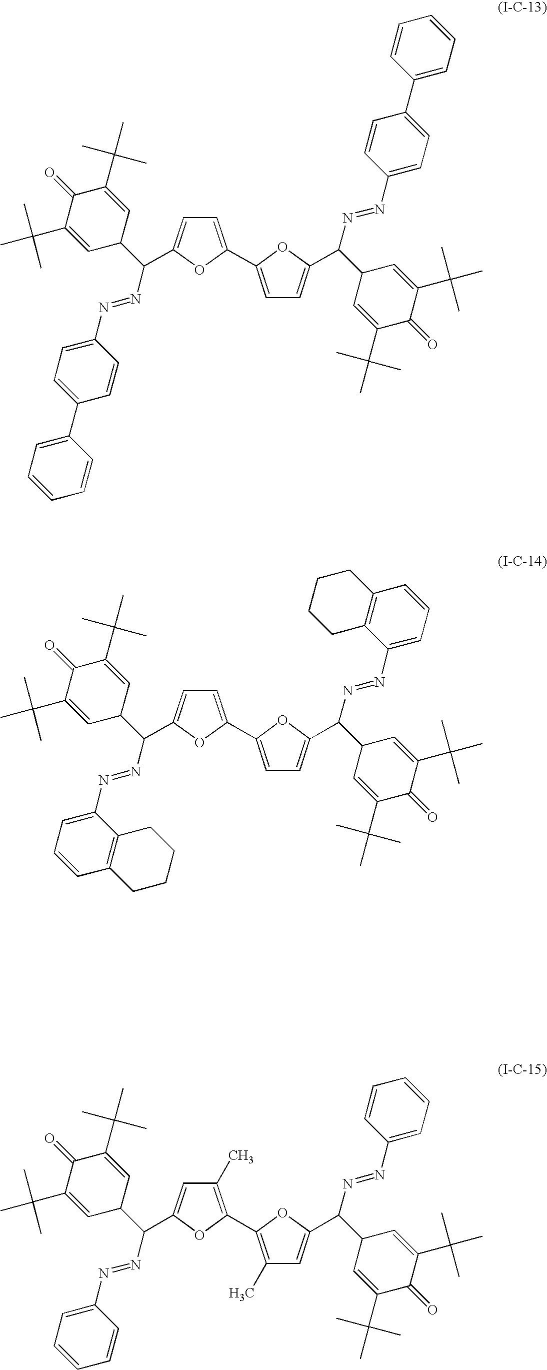

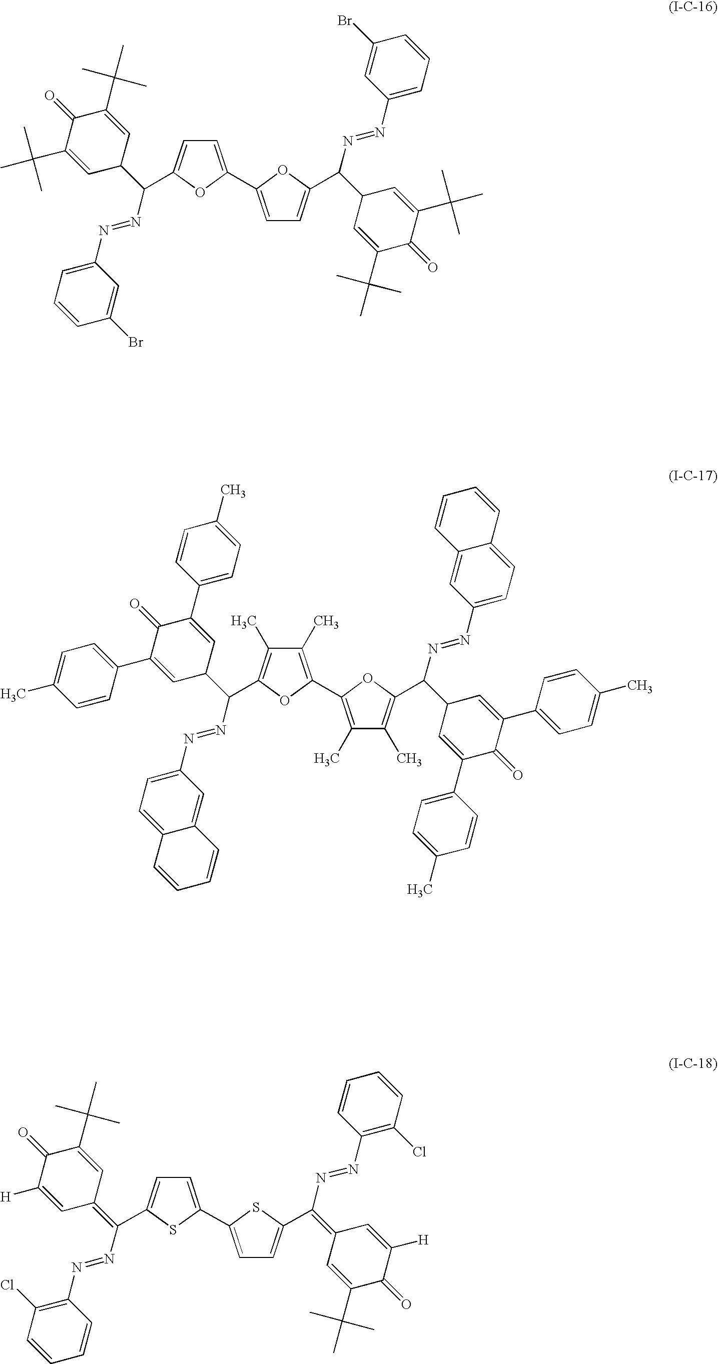

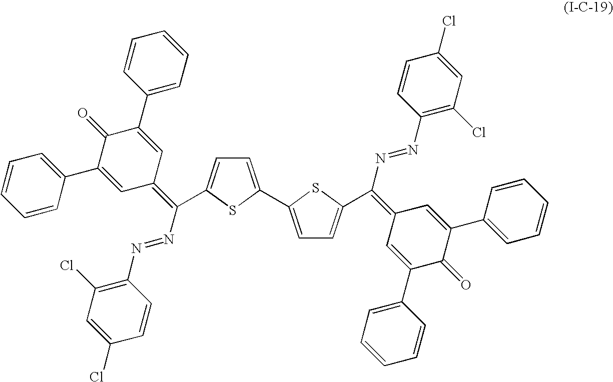

The compound represented by the foregoing general formula (I-A) according to invention can be, for example, synthesized according to the following reaction schemes (1-A) and (2-A). That is, first of all, as shown in the following reaction scheme (1-A), a compound represented by the structural formula (b-A) is synthesized from a compound represented by the structural formula (b′-A). Next, as shown in the following reaction scheme (2-A), this compound represented by the structural formula (b-A) is allowed to react with a compound represented by the structural formula (a-A) and/or a compound represented by the structural formula (a′-A) and an appropriate organometallic reagent (for example, magnesium), and thereafter, a protective group (TMS: trimethylsilyl group) is eliminated to synthesize a compound represented by the structural formula (c-A). In addition, this compound represented by the structural formula (c-A) is subjected to dehydration condensation with a compound represented by the structural formula (d-A) and/or a compound represented by the structural formula (d′-A), followed by oxidation with an appropriate catalyst (for example, lead dioxide (PbO₂)). Thus, the compound represented by the structural formula (I-A) can be synthesized. Incidentally, the term “TBAF” in the following reaction scheme (2-A) means tetrabutylammonium fluoride.

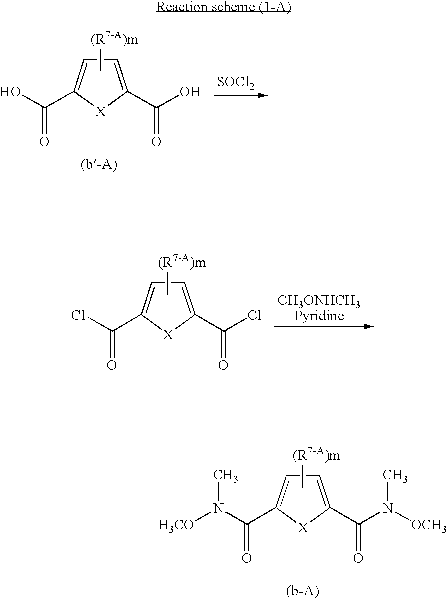

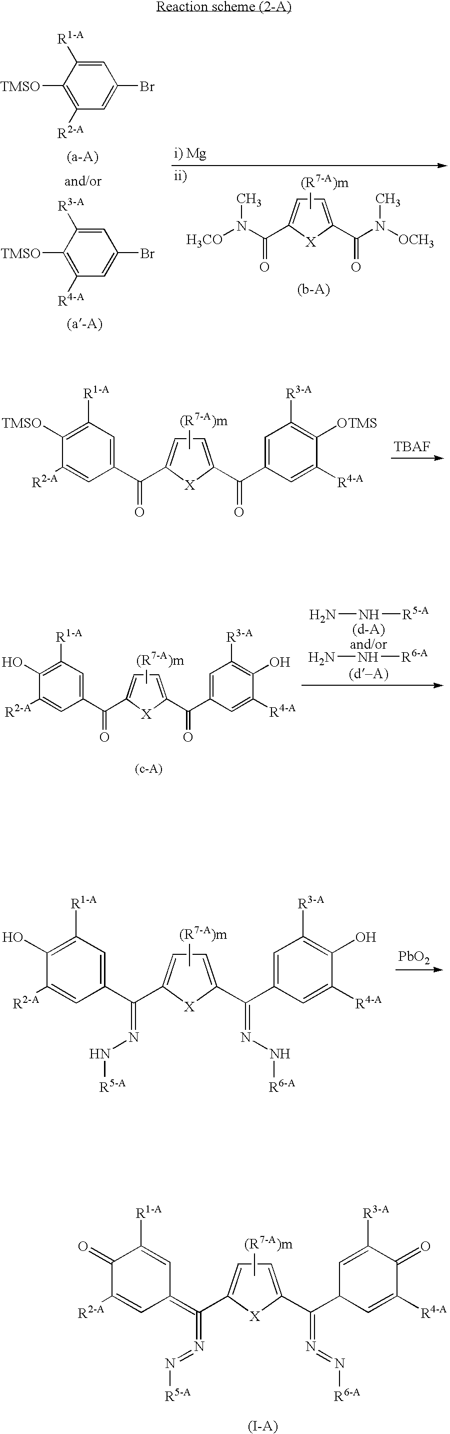

Furthermore, the compound represented by the foregoing general formula (I-B) according to invention can be, for example, synthesized according to the following reaction schemes (1-B) and (2-B). That is, first of all, as shown in the following reaction scheme (1-B), a compound represented by the structural formula (b-B) is synthesized from a compound represented by the structural formula (b′-B). Next, as shown in the following reaction scheme (2-B), the compound represented by the structural formula (b-B) is allowed to react with a compound represented by the structural formula (a-B) and/or a compound represented by the structural formula (a′-B) and an appropriate organometallic reagent (for example, magnesium), and thereafter, a protective group (TMS: trimethylsilyl group) is eliminated to synthesize a compound represented by the structural formula (c-B), followed by dehydration condensation with a compound represented by the structural formula (d-B) and/or a compound represented by the structural formula (d′-B) and subsequent oxidation with an appropriate catalyst (for example, lead dioxide (PbO₂)). Thus, the compound represented by the structural formula (I-B) can be synthesized. Incidentally, the term “TBAF” in the following reaction scheme (2-B) means tetrabutylammonium fluoride.

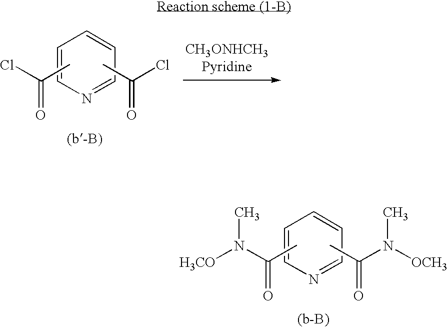

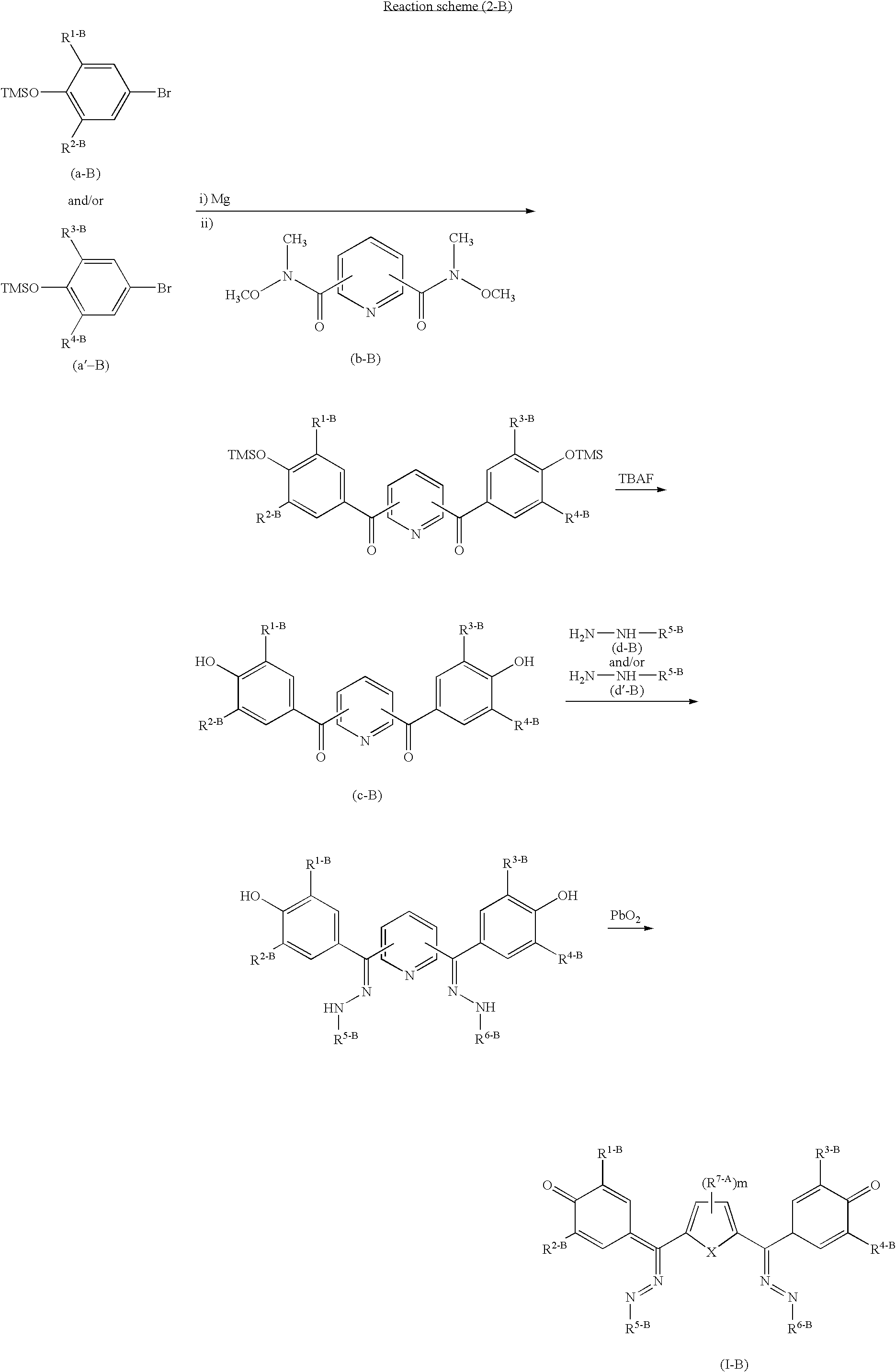

In addition, the compound represented by the foregoing general formula (I-C) according to invention can be, for example, synthesized according to the following reaction schemes (1-C) and (2-C). That is, first of all, as shown in the following reaction scheme (1-C), a compound represented by the structural formula (b-C) is synthesized from a compound represented by the structural formula (b′-C). Next, as shown in the following reaction scheme (2-C), the compound represented by the structural formula (b-C) is allowed to react with a compound represented by the structural formula (a-C) and/or a compound represented by the structural formula (a′-C) and an appropriate organometallic reagent (for example, magnesium), and thereafter, a protective group (TMS: trimethylsilyl group) is eliminated to synthesize a compound represented by the structural formula (c-C), followed by dehydration condensation with a compound represented by the structural formula (d-C) and/or a compound represented by the structural formula (d′-C) and subsequent oxidation with an appropriate catalyst (for example, lead dioxide (PbO₂)). Thus, the compound represented by the structural formula (I-C) can be synthesized. Incidentally, the term “TBAF” in the following reaction scheme (2-C) means tetrabutylammonium fluoride.

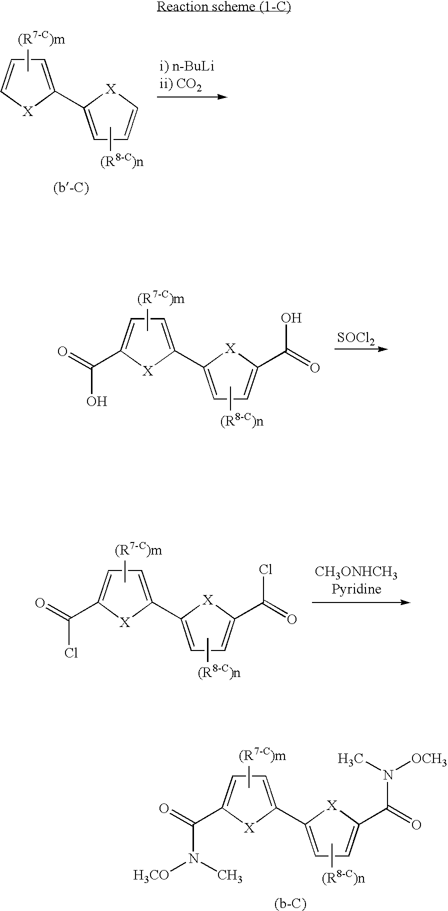

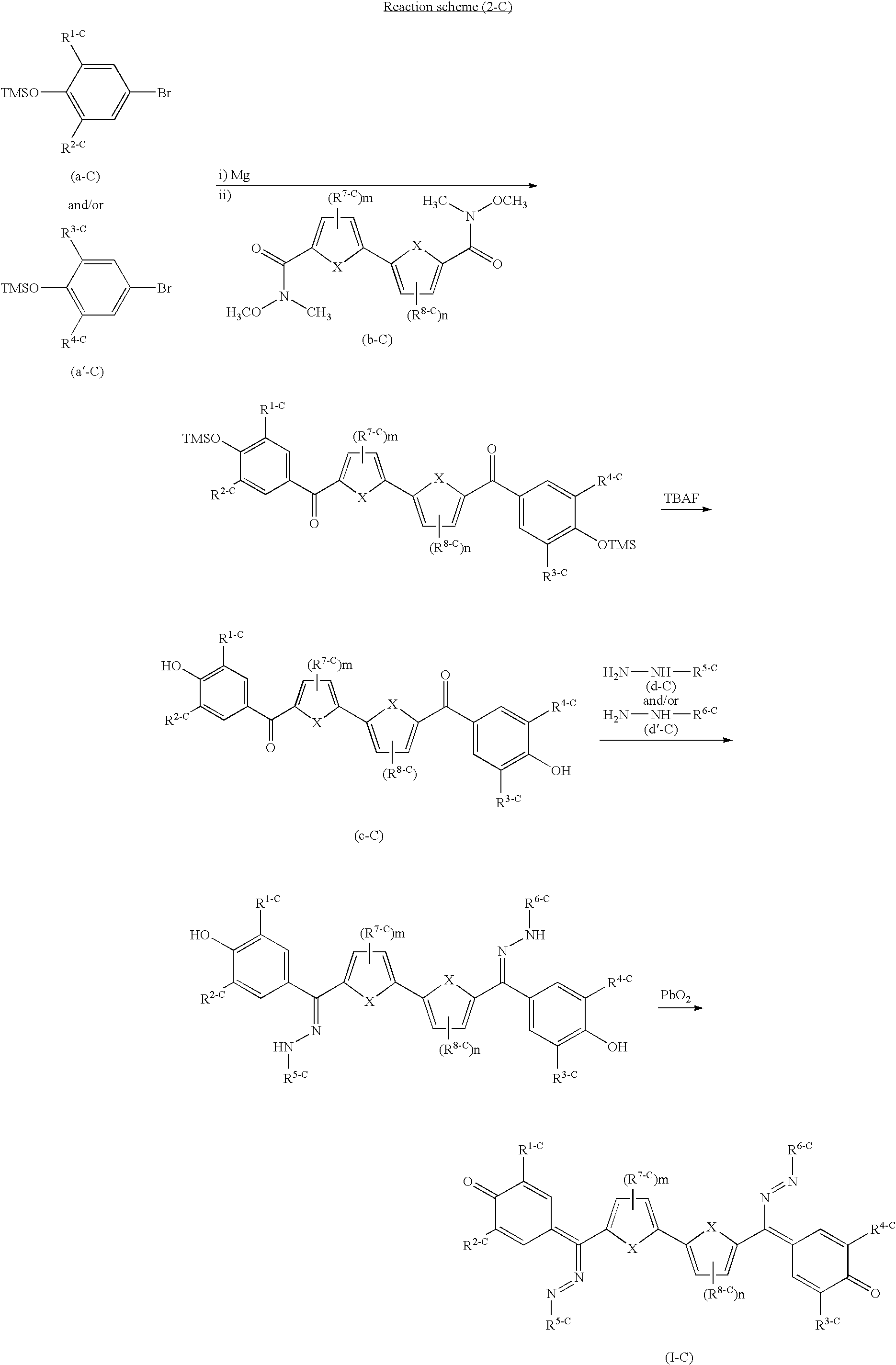

Embodiments of the electrophotographic photoreceptor of the invention will be hereunder described with reference to the accompanying drawings.

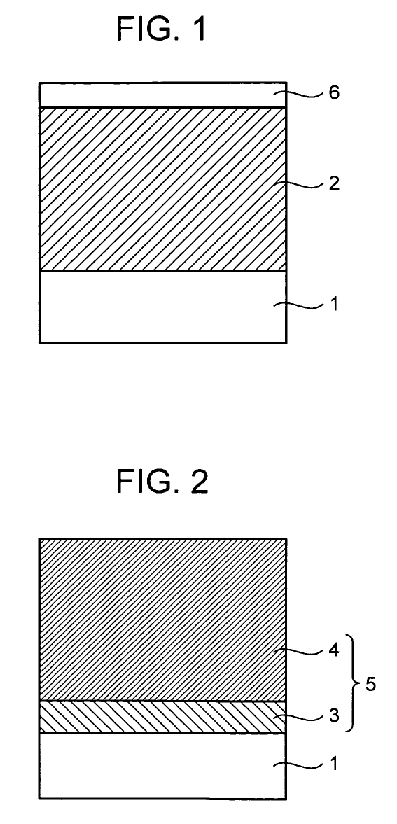

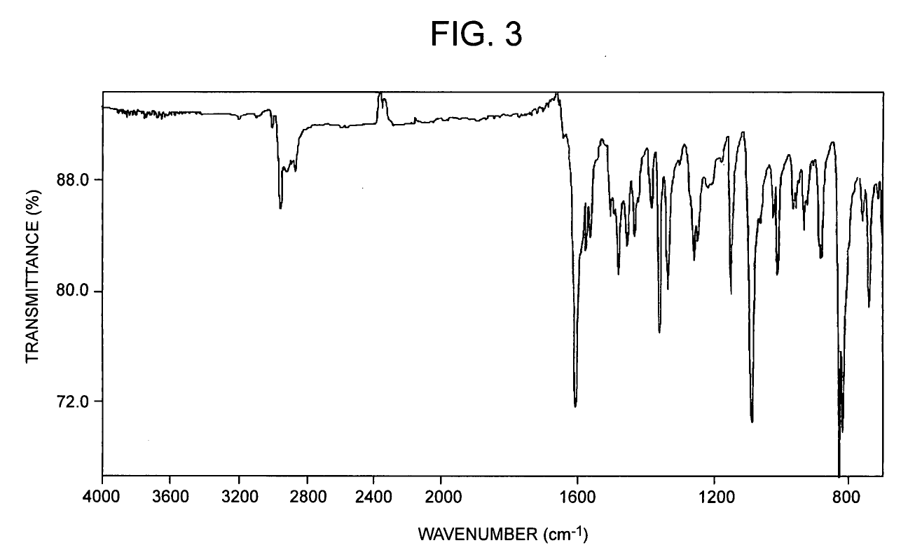

FIG. 1 and FIG. 2 are each a schematic cross-sectional view to show an example of the construction of the two kinds of photoreceptor that comprise the invention. Reference numeral 1 designates an electrically conductive substrate; reference numerals 2 and 5 designate a photosensitive layer; reference numeral 3 designates a charge generation layer; a reference numeral 4 designates a charge transport layer; and reference numeral 6 designates an optional covering layer, respectively. FIG. 1 shows one example of the construction of a so-called single-layer-type photoreceptor, in which the photosensitive layer 2 of a single layer containing a charge generation substance and a charge transport substance dispersed in a resin binder (binding agent) is provided on the electrically conductive substrate 1, and if desired, the covering layer (protective layer) 6 is laminated thereon as well. This single-layer-type photoreceptor can be prepared by dispersing a charge generation substance in a solution having a charge transport substance and a resin binder dissolved therein and coating this dispersion on an electrically conductive substrate. In addition, as the need arises, it is possible to coat thereover the covering layer 6, for example, an inorganic metal protective layer.

FIG. 2 shows one example of the construction of a so-called layered-type photoreceptor, in which the photosensitive layer 5 in which the charge generation layer 3, composed mainly of a charge generation substance and the charge transport layer 4 containing a charge transport substance are successively laminated, is provided on the electrically conductive substrate 1. This layered type photoreceptor can be prepared as follows: Either a charge generation substance is vacuum deposited on an electrically conductive substrate, or particles of the charge generation substance dispersed in a solvent or a resin binder are coated on the electrically conductive substrate. This is followed by drying to form the charge generation layer 3. Subsequently, a solution having a charge transport substance and a resin binder dissolved therein are coated on the charge generation layer 3, followed by drying, to form the charge transport layer 4.

Furthermore, in a photoreceptor of either type, it is possible to provide an undercoat layer (not illustrated in the drawings) between the electrically conductive substrate and the photosensitive layer. Such an undercoat layer can be provided depending on the situation for the purpose of preventing injection of an unnecessary charge from the electrically conductive substrate into the photosensitive layer or enhancing covering of a defect on the substrate surface or adhesion of the photosensitive layer or the like. The undercoat layer would be comprised of a layer containing a resin as the major component or an oxidized film made of alumite or the like.

Incidentally, any type of the photoreceptor of the invention contains, as the charge transport substance, at least one compound having electron transport properties according to the invention as represented by the foregoing general formula (1).

A preferred embodiment of the photoreceptor of the invention will be hereunder described with reference to the layered type photoreceptor as shown in FIG. 2, but it should not be construed that the invention is limited to the following specific example.

The electrically conductive substrate 1 serves as an electrode of the photoreceptor and simultaneously functions as a support for other respective layers, and may be in any of a cylindrical, plate-like or film-like form. With respect to the type of material, metals such as aluminum, stainless steel, and nickel, glasses or resins which have been subjected to an electrically conductive treatment, and the like can be employed.

As described previously, the charge generation layer 3 is formed by coating a dispersion material having particles of a charge generation substance in a resin binder, or formed vacuum deposition or other methods, and receives light to generate a charge. Also, it is important that its efficiency of charge generation is high, and at the same time, injection properties of the generated charge into the charge transport layer 4 are important. It is desired that the electric field dependency is low and that the injection is good even in a low electric field.

As the charge generation substance, the following are examples substances that can be employed:

phthalocyanine compounds such as metal-free phthalocyanine and titanyl phthalocyanine,

pigments or dyes such as a variety of azo, quinone, indigo, cyanine, squarylium, azulenium and pyrylium compounds, and

selenium or selenium compounds. A suitable substance can be chosen according to the wavelength region of light from an exposure light source to be used for the image formation. Since the charge generation layer is only required to have a charge generation function, its thickness is determined by the coefficient of light absorption of the charge generation substance and is generally not more than 5 μm, and preferably not more than 2 μm. In addition, in the charge generation layer, comprised mainly of a charge generation substance, it is also possible to add and use a charge transport substance, etc.

As the resin binder for the charge generation layer 3, polycarbonates, polyesters, polyamides, polyurethanes, vinyl chloride resins, phenoxy resins, polyvinyl butyral, diallyl phthalate resin, polymers or copolymers of methacrylic esters, and the like can be properly combined and used.

The charge transport layer 4 is a coating film having a charge transport substance dispersed in a resin binder. It exhibits a function to hold a charge of the photoreceptor as an insulator layer in a dark place and to transport a charge as injected from the charge generation layer at the time of receiving light. As described previously, in the invention, although it is required that at least one compound having electron transport properties according to the invention as represented by the foregoing general formula (I) is contained in the charge transport layer 4 as such a charge transport substance, other charge transport substances may be contained in this layer. The amount of this compound contained in the charge transport layer according to the invention is preferably from 10 to 60% by weight, and more preferably from 15 to 50% by weight [[4]].

As the resin binder for the charge transport layer 4, polycarbonates, polyesters, polystyrenes, polymers or copolymers of methacrylic esters, and the like can be used.

For the purpose of preventing deterioration due to ozone, and the resulting hindrance to the desired performance of the photoreceptor, from occurring or other purposes, it is also possible to include, in the charge transport layer 4, an antioxidant such as amine-based, phenol-based, sulfur-based, phosphite-based, and phosphorus-based antioxidants.

The covering layer 6 as shown in FIG. 1 is required to receive a charge of corona discharge and hold it in a dark place, transmit light to which the photosensitive layer is sensitive, and at the time of exposure, transmit light and make it arrive at the photosensitive layer, and upon receipt of the injection of a generated charge, neutralize the surface charge, thereby to extinguish it. As a material of the covering layer, an organic insulating film forming material such as a polyester or polyamide can be applied as an organic material. Furthermore, it is possible to use a mixture of such an organic material with an inorganic material such as glass or SiO₂, and further with a material capable of reducing electrical resistance such as a metal or metal oxide. As the inorganic material, it is possible to apply a layer as prepared by film formation by the vapor phase epitaxy, such as an amorphous silicon-carbon (SiC) composite film covering layer. It is desired that the covering layer is transparent as far as possible in a wavelength region of the absorption maximum of light of the charge generation substance as described previously.

Though the film thickness of the covering layer itself varies depending upon the blending composition of the covering layer, it can be arbitrarily set within the range where at the time of repeated continuous use, adverse influences such as an increase of the residual potential are not revealed.

Incidentally, even in the case of the single layer type photoreceptor as shown in FIG. 1, it is required to contain at least one compound having electron transport properties according to the invention as represented by the foregoing general formula (I) in the photosensitive layer 2. With respect to other materials, those which are the same as in the foregoing layered type photoreceptor can be used, and there are no particular limitations therein. Suitably, a hole transport substance is contained in the photosensitive layer together with the compound of the foregoing general formula (I) as the charge transport substance. As the hole transport substance, benzidine derivatives, triphenylamine derivatives, and the like are preferable. In this case, with respect to the amounts of these substances, the amount of the compound according to the invention is suitably from 10 to 60% by weight, and more preferably from 15 to 50% by weight, and the amount of the hole transport substance is suitably from 10 to 60% by weight, and more suitably from 20 to 50% by weight based on all of the materials to be contained in the photosensitive layer forming coating film.

Furthermore, in the surface layer of the photoreceptor (the covering layer in the case where the covering layer is provided, and the photosensitive layer in the outermost side in the case where the covering layer is not provided), it is possible to add a silicone oil as a leveling material; and for the purpose of imparting lubricating properties, it is possible to contain a silicon or fluorine-containing polymer such as a silicone oil, a fluorine resin fine particle material such as tetrafluoroethylene resins, a silicone resin fine particle material, and a fluorine based comb type graft polymer.

### EXAMPLES

Examples of the invention will be hereunder described.

### Synthesis Example 1-A

### Synthesis of Compound of the Foregoing Specific Example (I-A-3)

A compound represented by the foregoing specific example (I-A-3) was synthesized according to the following reaction schemes (1-A-1) and (2-A-1).

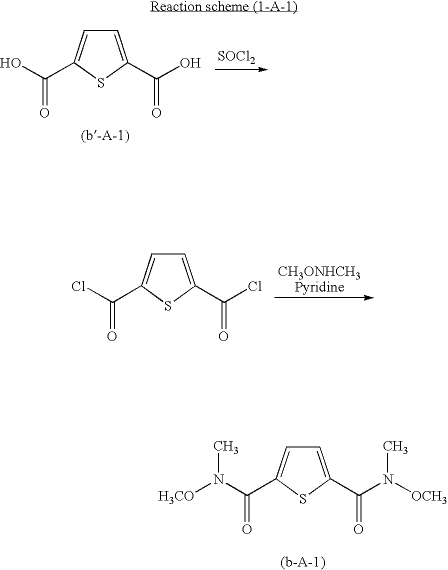

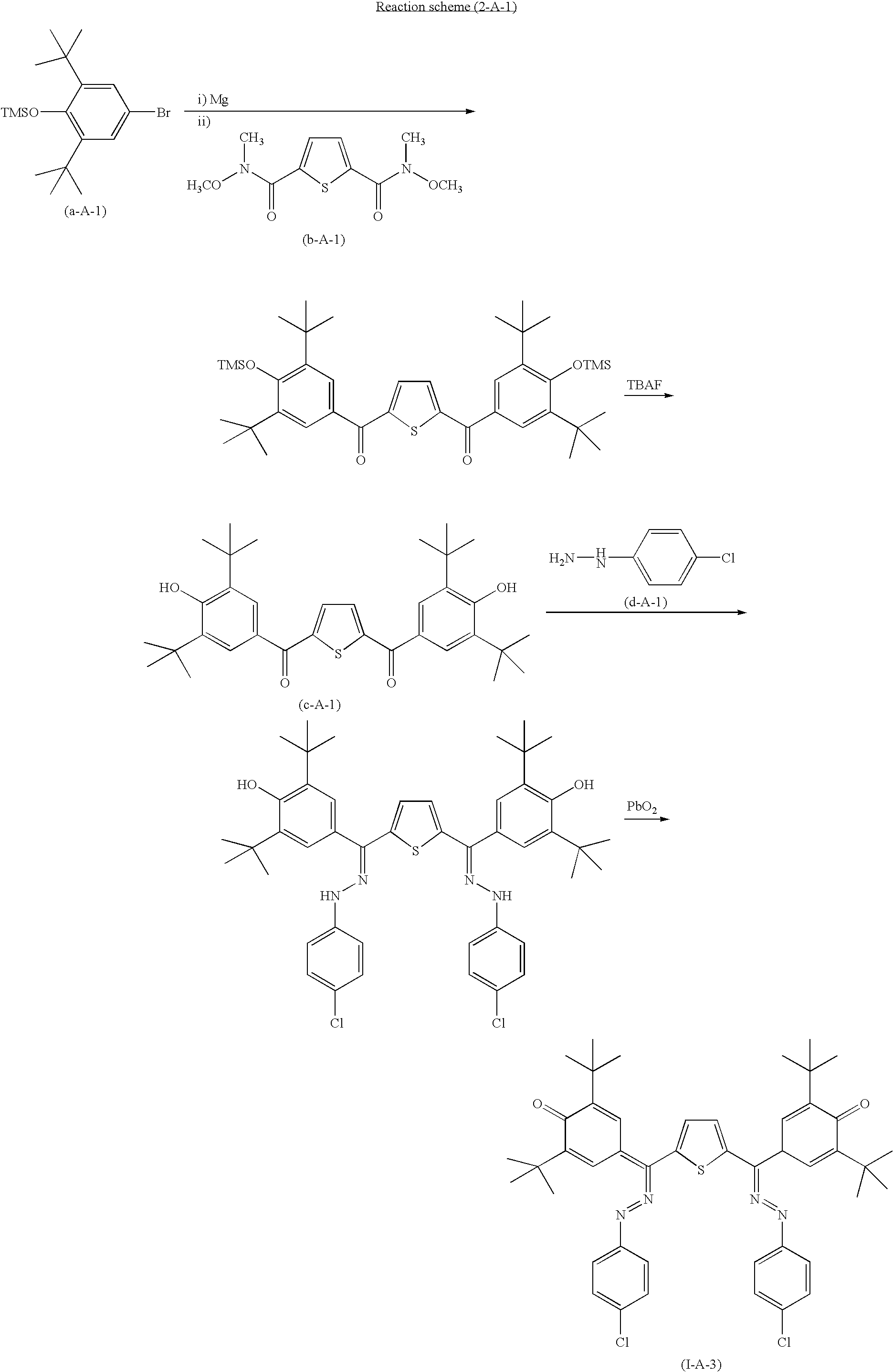

To 100 mmoles (17.2 g) of thiophenedicarboxylic acid (the foregoing structural formula (b′-A-1)), 250 mmoles (29.8 g) of thionyl chloride was added, and the mixture was refluxed under heat. Thereafter, the excessive thionyl chloride was evaporated in vacuo to obtain an oily substance. To a dichloromethane solution of this compound and 200 mmoles (19.5 g) of N,O-dimethylhydroxyamine hydrochloride, 600 mmoles (47.5 g) of pyridine were added in a nitrogen atmosphere at room temperature, and the mixture was stirred for 2 hours. Thereafter, the reaction solution was poured into aqueous hydrochloric acid and extracted with dichloromethane, followed by concentration. There was thus obtained, as a crude product, a compound represented by the foregoing structural formula (b-A-1) in a yield of 21.1 g (81.7%).

Next, to a THF solution of 30 mmoles (7.75 g) of the compound represented by the foregoing structural formula (b-A-1), a Grignard reagent as prepared from 99 mmoles (2.41 g) of magnesium and a THF solution of 90 mmoles (32.2 g) of 4-bromo-2,6-di-t-butyl-1-(trimethylsiloxy)benzene (a-A-1) was added dropwise, and the mixture was stirred at room temperature for 3 hours. Thereafter, a small amount of a 1N hydrochloric acid aqueous solution was added to stop the reaction. In addition, 90 mmoles (90 mL) of a THF solution of 1.0 M tetrabutylammonium fluoride (TBAF) was added, followed by stirring. The reaction solution was poured into aqueous hydrochloric acid and extracted with dichloromethane, followed by concentration. There was thus obtained, as a crude product, a compound represented by the foregoing structural formula (c-A-1) in a yield of 8.12 g (49.2%).

In addition, 10 mmoles (5.48 g) of the compound represented by the foregoing structural formula (c-A-1) and 30 mmoles (5.37 g) of p-chlorophenylhydrazine hydrochloride (the foregoing structural formula (d-A-1)) were dissolved in a mixed solution of pyridine and ethanol, and the mixture was refluxed under heat. The reaction solution was poured into aqueous hydrochloric acid and extracted with dichloromethane, followed by concentration. Thereafter, the resulting product was purified by column chromatography to obtain a crude product.

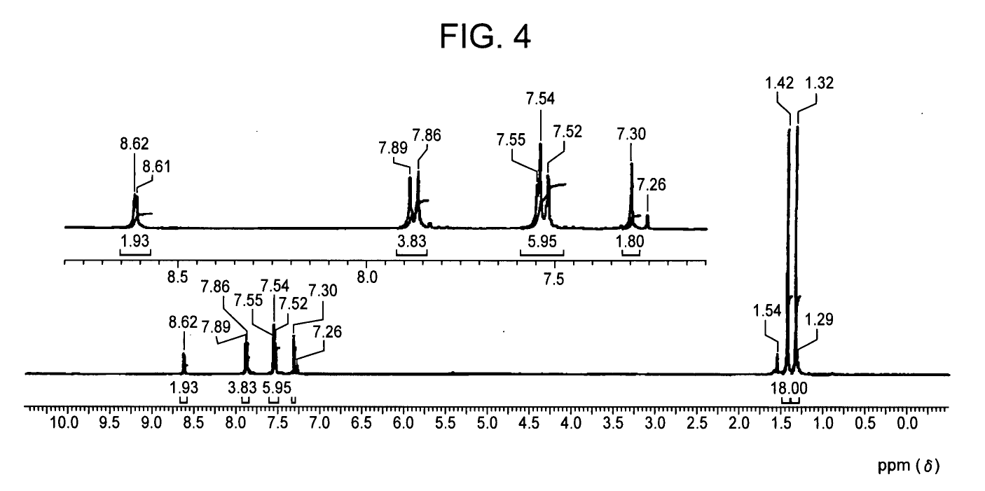

To a chloroform solution of the foregoing crude product, 30 mmoles (7.18 g) of lead dioxide (PbO₂) was added at room temperature, and the mixture was stirred. After filtering out the residue, the reaction solution was concentrated, and the resulting solid was recrystallized from hexane to obtain a compound represented by the foregoing structural formula (I-A-3). Yield: 4.9 g (percent yield: 61.7%), MS: m/z 792 (M+). Incidentally, the entire yield was 24.8%. The IR spectrum and ¹H-NMR spectrum of this compound of the structural formula (I-A-3) are shown in FIG. 3 and FIG. 4, respectively.

### Synthesis Example 2-A

### Synthesis of Compound of the Foregoing Specific Example (I-A-5)

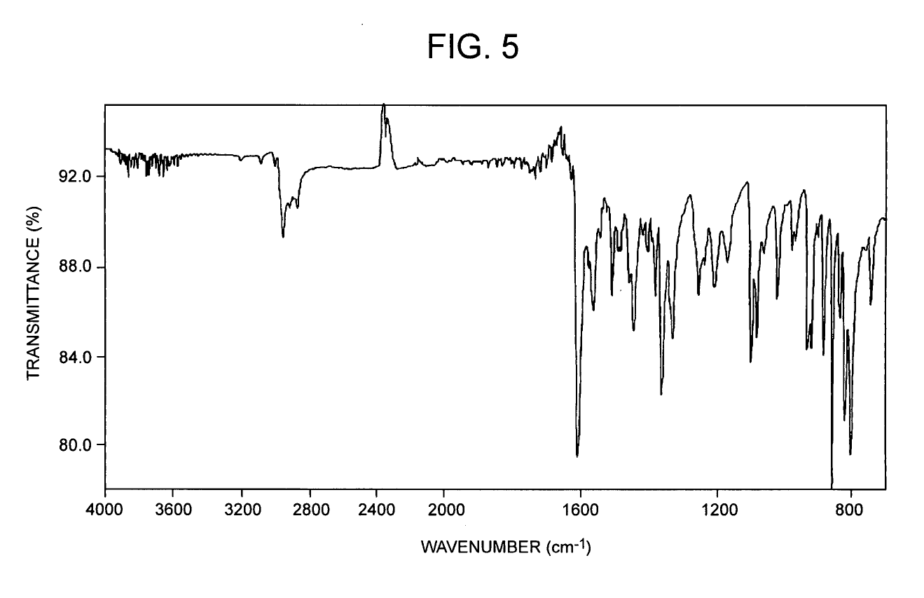

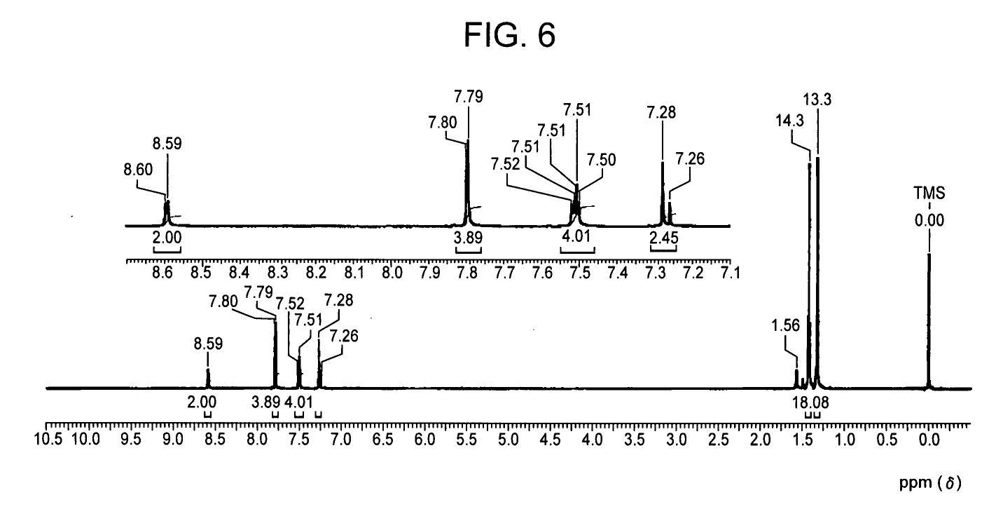

A compound represented by the foregoing structural formula (I-A-5) was obtained by following the same operations as in Synthesis Example 1-A, except for changing the 4-chlorophenylhydrazine as used in Synthesis Example 1-A to 3,5-dichlorophenylhydrazine. Entire yield: 18.9%, MS: m/z 860 (M+). The IR spectrum and ¹H-NMR spectrum of this compound of the structural formula (I-A-5) are shown in FIG. 5 and FIG. 6, respectively.

Incidentally, the 4-bromo-2,6-di-tert-butyl-1-(trimethylsiloxy)benzene as used in the foregoing Synthesis Examples can be, for example, synthesized by a known method as described in the foregoing Patent Document 15 and so on.

### Photoreceptor Example 1-A

A plate-like photoreceptor for evaluating electrical characteristics and a drum-like photoreceptor for evaluating printing were prepared, respectively.

A solution for an undercoat layer as prepared in the following manner was coated on the external surface of each of an aluminum plate (3 cm×10 cm, thickness: 1 mm) and an aluminum drum (outer diameter: 30 mmφ, length: 247.5 mm, thickness: 0.75 mm) by the dip coating method and then dried at 100° C. for 60 minutes to remove the solvent. There was thus formed an undercoat layer having a film thickness of 0.3 μm.

(Preparation of Solution for Undercoat Layer)

A-a1) Soluble nylon (AMILAN CM8000, manufactured by Toray Industries, Inc.): 3 parts by weight (30 g)

The foregoing material A-a1) for the undercoat layer was stirred together with 97 parts by weight (970 g) of a mixed solvent of methanol/methylene chloride (5 vol./5 vol.) and dissolved to prepare a solution for the undercoat layer.

Next, on this undercoat layer, a dispersion for single layer type photosensitive layer as prepared in the following manner was coated by the dip coating method for the plate-like photoreceptor and by the ring coating method for the drum-like photoreceptor, respectively and then dried at 100° C. for 60 minutes to remove the solvent. Thus, a single layer type photosensitive layer having a film thickness of 25 μm was formed, from which an electrophotographic photoreceptor was then prepared.

(Preparation of Dispersion for Single Layer Type Photosensitive Layer)

A-b1) Charge generation substance: X-type metal-free phthalocyanine (see FIG. 2 of JP-A-2001-228637): 0.3 parts by weight (0.15 g)

A-b2) Hole transport substance: triphenylamine compound represented by the following structural formula (HT1-101) ((HT1-101) of JP-A-2000-314969): 7 parts by weight (3.5 g)

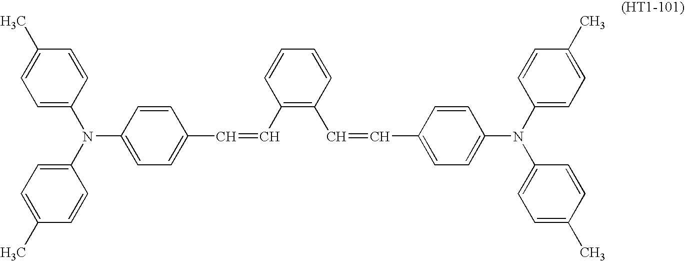

A-b3) Electron transport substance: Compound represented by the foregoing structural formula (1-A-3): 3 parts by weight (1.5 g)

A-b4) Antioxidant: 3,5-Di-tert-4-hydroxytoluene (BHT) (manufactured by Tokyo Kasei Kogyo Co., Ltd.): 1 part by weight (0.5 g)

A-b5) Silicone oil: KF-50 (manufactured by Shin-Etsu Chemical Co., Ltd.): 0.01 parts by weight (0.005 g)

A-b6) Binder resin: Bisphenol Z type polycarbonate resin (PANLITE TS2020, manufactured by Teijin Chemicals Ltd.) ((BD1-1) of JP-A-2000-314969): 10 parts by weight (5 g).

In a 100-mL plastic bottle, the foregoing materials A-b1) to A-b6) for a photosensitive layer were charged together with 100 parts by weight (50 g) of a methylene chloride solvent and 50 g of stainless steel beads (3 mmφ), and the mixture was subjected to a dispersing treatment for 60 minutes by a paint conditioner Model 5400 (Red Devil Equipment Co., U.S.A.). Thereafter, the stainless steel beads were separated to prepare a dispersion for a single layer type photosensitive layer.

### Photoreceptor Example 2-A

A single layer type photoreceptor was prepared in the same manner as in Photoreceptor Example 1-A, except that in the composition of the dispersion for photosensitive layer as used in Photoreceptor Example 1-A, 3 parts by weight of the compound represented by the foregoing structural formula (1-A-3) as the electron transport substance was replaced by 3 parts by weight of the compound represented by the foregoing structural formula (1-A-5).

### Photoreceptor Example 3-A

A single layer type photoreceptor was prepared in the same manner as in Photoreceptor Example 1-A, except that in the composition of the dispersion for photosensitive layer as used in Photoreceptor Example 1-A, 3 parts by weight of the compound represented by the foregoing structural formula (I-A-3) as the electron transport substance was replaced by 3 parts by weight of the compound represented by the foregoing structural formula (I-A-26).

### Photoreceptor Example 4-A

A single layer type photoreceptor was prepared in the same manner as in Photoreceptor Example 1-A, except that in the composition of the dispersion for photosensitive layer as used in Photoreceptor Example 1-A, 3 parts by weight of the compound represented by the foregoing structural formula (I-A-3) as the electron transport substance was replaced by 3 parts by weight of the compound represented by the foregoing structural formula (I-A-30).

### Photoreceptor Example 5-A

A single layer type photoreceptor was prepared in the same manner as in Photoreceptor Example 1-A, except that in the composition of the dispersion for photosensitive layer as used in Photoreceptor Example 1-A, 7 parts by weight of the triphenylamine compound represented by the foregoing structural formula (HT1-101) as the hole transport substance was replaced by 7 parts by weight of a styryl compound represented by the following structural formula (HT2-2) ((HT2-2) of JP-A-2000-314969).

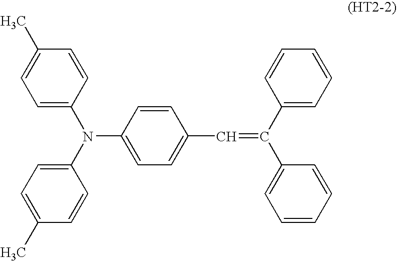

### Photoreceptor Example 6-A

A single layer type photoreceptor was prepared in the same manner as in Photoreceptor Example 1-A, except that in the composition of the dispersion for photosensitive layer as used in Photoreceptor Example 1-A, 7 parts by weight of the triphenylamine compound represented by the foregoing structural formula (HT1-101) as the hole transport substance was replaced by 7 parts by weight of a benzidine derivative represented by the following structural formula (HT-11) ((HT-11) of JP-A-2000-314969).

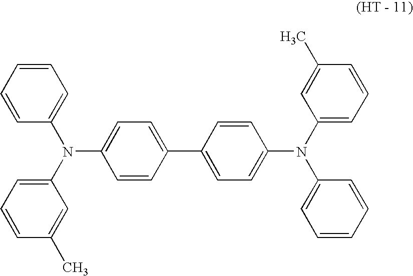

### Photoreceptor Example 7-A

A single layer type photoreceptor was prepared in the same manner as in Photoreceptor Example 1-A, except that in the composition of the dispersion for photosensitive layer as used in Photoreceptor Example 1-A, 0.3 parts by weight of the X-type metal-free phthalocyanine as the charge generation substance was replaced by 0.2 parts by weight of Y-type titanyl phthalocyanine (see FIG. 4 of JP-A-2001-228637).

### Photoreceptor Example 8-A

A single layer type photoreceptor was prepared in the same manner as in Photoreceptor Example 1-A, except that in the composition of the dispersion for photosensitive layer as used in Photoreceptor Example 1-A, 0.3 parts by weight of the X-type metal-free phthalocyanine as the charge generation substance was replaced by a bisazo pigment represented by the following formula (CG1-1).

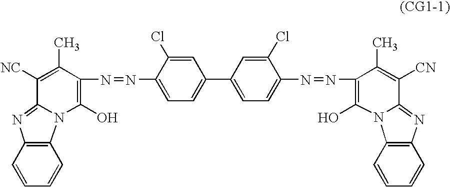

### Photoreceptor Comparative Example 1-A

A single layer type photoreceptor was prepared in the same manner as in Photoreceptor Example 1-A, except that in the composition of the dispersion for photosensitive layer as used in Photoreceptor Example 1-A, 3 parts by weight of the compound represented by the foregoing structural formula (1-A-3) as the electron transport substance was replaced by 3 parts by weight of a compound represented by the following structural formula (ET4-1) ((ET4-1) of JP-A-2000-314969).

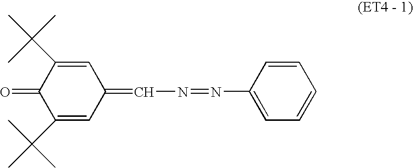

### Evaluation of Photoreceptors of Photoreceptor Examples 1-A to 8-A and Photoreceptor Comparative Example 1

Using a plate-like photoreceptor for evaluating electrical characteristics, the evaluation was carried out by an electrostatic copying paper analyzer EPA-8100 as manufactured by Kawaguchi Electric Works Co., Ltd.

First of all, the photoreceptor was charged in a dark place in an atmosphere at a temperature of 23° C. and a humidity of 45% such that the surface potential became about +700 V, and a retention rate Vₖ₅ of the surface potential after a lapse of 5 seconds was determined according to the following expression.
Retention rate Vₖ₅(%)=(V₅/V₀)×100

V₀: Surface potential immediately after charging

V₅: Surface potential 5 seconds after charging

Next, the surface potential was changed to +600 V, monochromic light of 1.0 μW/cm² resulting from separation of light of a halogen lamp into 780 nm (but into 550 nm with respect to the Photoreceptor Example 8-A) was exposed for 5 seconds, the amount of exposure required until the surface potential became one-half (+300 V) was determined as a sensitivity E_{1/2} (μJ/cm²), and the surface potential 5 seconds after the exposure was determined as a residual potential Vᵣ (V).

The evaluation results are shown in the following Table 1.

TABLE 1

| | Retention | Sensitivity | Residual |
|---|---|---|---|
| | rate | E_{1/2} | potential |
| | Vₖ₅ (%) | (μJ/cm²) | Vᵣ (V) |
| Photoreceptor Example 1-A | 85.6 | 0.29 | 24 |
| Photoreceptor Example 2-A | 82.2 | 0.37 | 31 |
| Photoreceptor Example 3-A | 83.5 | 0.35 | 33 |
| Photoreceptor Example 4-A | 84.4 | 0.40 | 39 |
| Photoreceptor Example 5-A | 81.5 | 0.32 | 36 |
| Photoreceptor Example 6-A | 80.9 | 0.33 | 38 |
| Photoreceptor Example 7-A | 83.9 | 0.31 | 28 |
| Photoreceptor Example 8-A *⁾ | 82.7 | 0.49 | 54 |
| Photoreceptor Comparative | 80.3 | 0.53 | 84 |
| Example 1-A | | | |

*⁾ Light at 550 nm was employed for exposure only in Example 8-A.

Furthermore, a drum-like photoreceptor (30 mmφ) was used for evaluating the durability by actual printing. Each drum-like photoreceptor was installed in a laser printer HL-1240 as manufactured by Brother Industries, Ltd. and printed with a black solid image, a white solid image, and a half-tone image, respectively in an atmosphere at a temperature of 22° C. and a humidity of 44%. Subsequently, 5,000 sheets were printed with an image having a rate of printing (page coverage) of about 5%. Thereafter, a black solid image, a white solid image, and a half-tone image were again printed, respectively, thereby evaluating the image after printing 5,000 sheets.

As a result, in the photoreceptors of Photoreceptor Examples 1-A to 7-A, a good image was obtained in both the initial image and the image after printing 5,000 sheets.

Incidentally, with respect to Photoreceptor Example 8-A, the photoreceptor did not have sufficient sensitivity in a laser wavelength region (in the vicinity of 780 nm) of the used laser printer and therefore, it was noted that this photoreceptor is not suitable for this laser printer.

### Synthesis Example 1-B

### Synthesis of Compound of the Foregoing Specific Example (I-B-4)

A compound of the foregoing specific example (I-B-4) was synthesized according to the following reaction schemes (1-B-1) and (2-B-1).

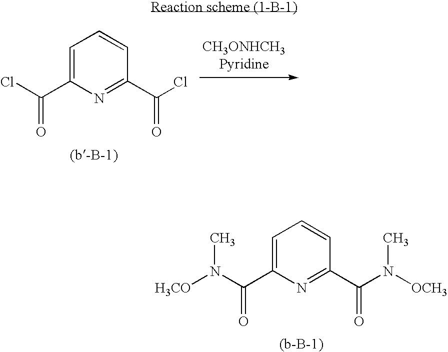

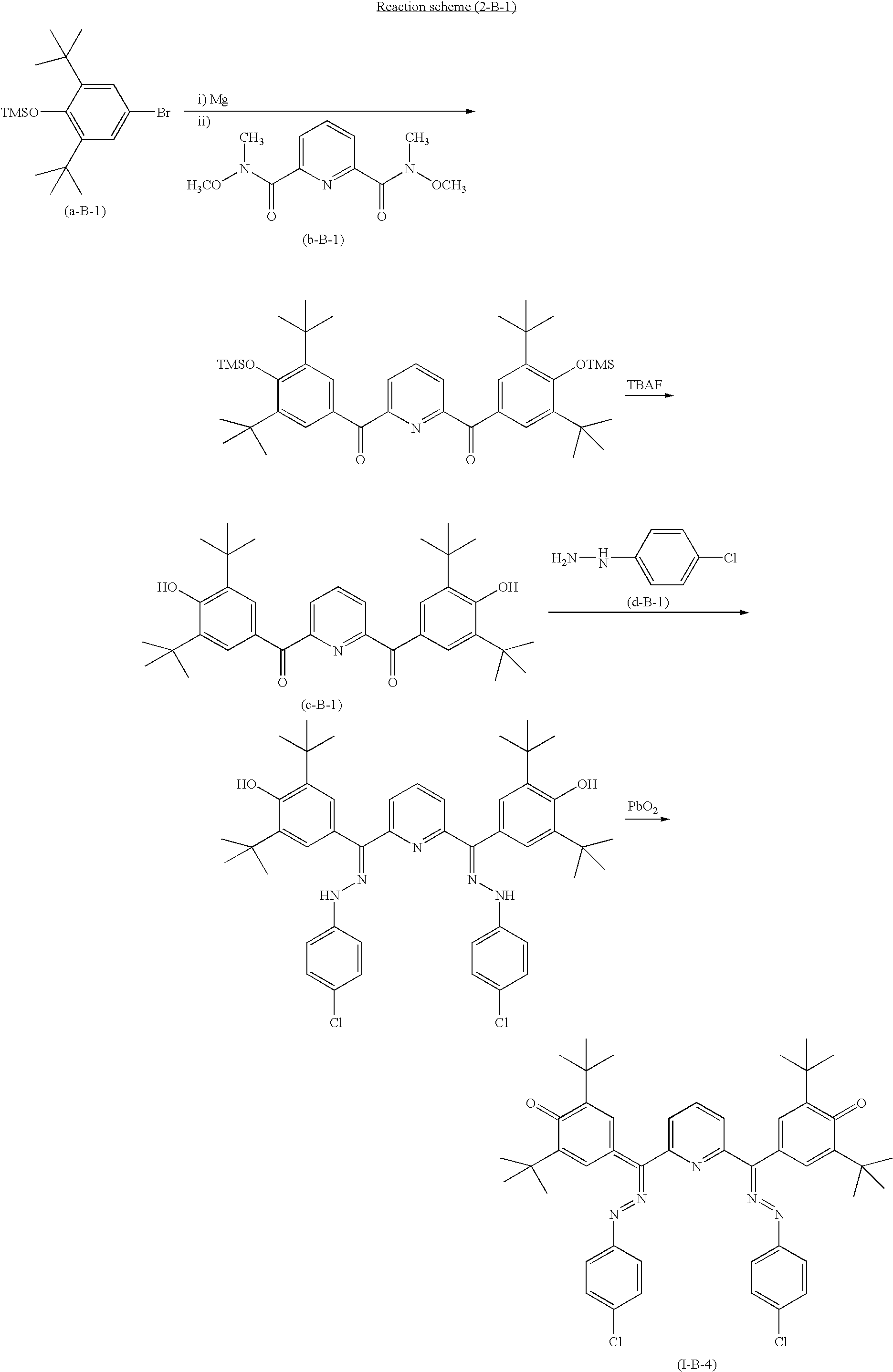

In a three-necked flask, 480 mmoles (38.0 g) of pyridine was added in a dichloromethane solution of 100 mmoles (20.4 g) of 2,6-pyridinecarbonyl dichloride (the foregoing structural formula (b′-B-1)) and 230 mmoles (22.4 g) of N,O-dimethylhydroxyamine hydrochloride at room temperature in a nitrogen atmosphere, and the mixture was stirred for 2 hours. Thereafter, the reaction solution was poured into aqueous hydrochloric acid and extracted with dichloromethane, followed by concentration. There was thus obtained, as a crude product, a compound represented by the foregoing structural formula (b-B-1) in a yield of 22.8 g (89.9%)

Next, to a THF solution of 30 mmoles (7.6 g) of the compound represented by the foregoing structural formula (b-B-1), a Grignard reagent as prepared from 99 mmoles (2.4 g) of magnesium and a THF solution of 90 mmoles (32.2 g) of 4-bromo-2,6-di-t-butyl-1-(trimethylsiloxy)benzene (the foregoing structural formula (a-B-1)) was added dropwise, and the mixture was stirred at room temperature for 3 hours. Thereafter, a small amount of a 1N hydrochloric acid aqueous solution was added to stop the reaction. In addition, 90 mmoles (90 mL) of a THF solution of 1.0 M tetrabutylammonium fluoride (TBAF) was added, followed by stirring. The reaction solution was poured into aqueous hydrochloric acid and extracted with dichloromethane, followed by concentration. There was thus obtained, as a crude product, a compound represented by the foregoing structural formula (c-B-1) in a yield of 12.8 g (78.5%).

In addition, 10 mmoles (5.4 g) of the compound represented by the foregoing structural formula (c-B-1) and 30 mmoles (5.4 g) of 4-chlorophenylhydrazine (the foregoing structural formula (d-B-1)) were dissolved in pyridine, and the mixture was refluxed under heat. The reaction solution was poured into aqueous hydrochloric acid and extracted with dichloromethane, followed by concentration. Thereafter, the resulting product was purified by column chromatography to obtain a crude product.

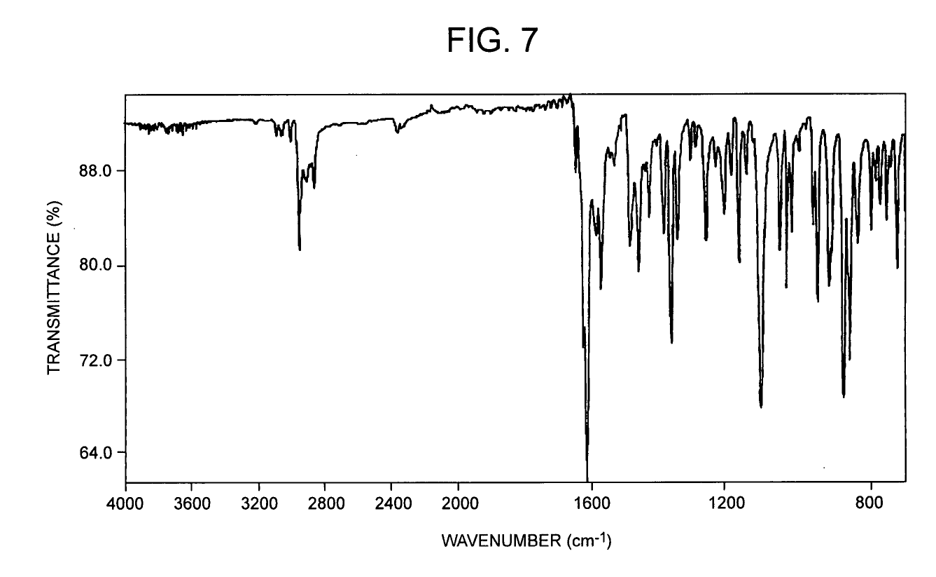

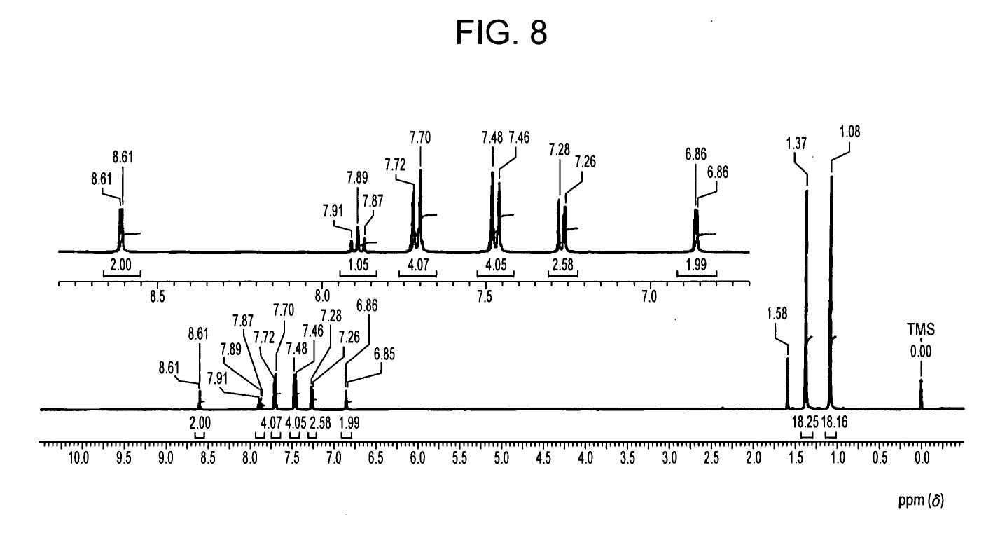

To a chloroform solution of the foregoing crude product, 30 mmoles (7.2 g) of lead dioxide (PbO₂) was added at room temperature, and the mixture was stirred. After filtering out the residue, the reaction solution was concentrated, and the resulting solid was recrystallized from hexane to obtain a compound represented by the foregoing structural formula (I-B4). Yield: 3.3 g (percent yield: 41.8%), MS: m/z 787 (M+). Incidentally, the entire yield was 29.5%. The IR spectrum and ¹H-NMR spectrum of this compound of the structural formula (I-B-4) are shown in FIG. 7 and FIG. 8, respectively.

Incidentally, the 4-bromo-2,6-di-tert-butyl-1-(trimethylsiloxy)benzene (the foregoing structural formula (a-B-1)) as used above can be, for example, synthesized by a known method as described in JP-A-2001-222122 and so on.

### Synthesis Example 2-B

### Synthesis of Compound of the foregoing specific Example (I-B-7)

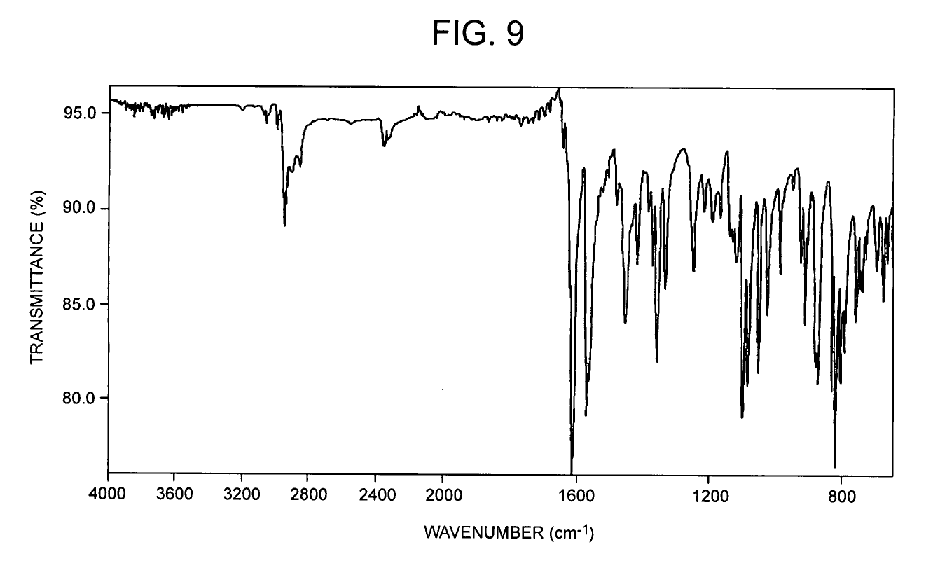

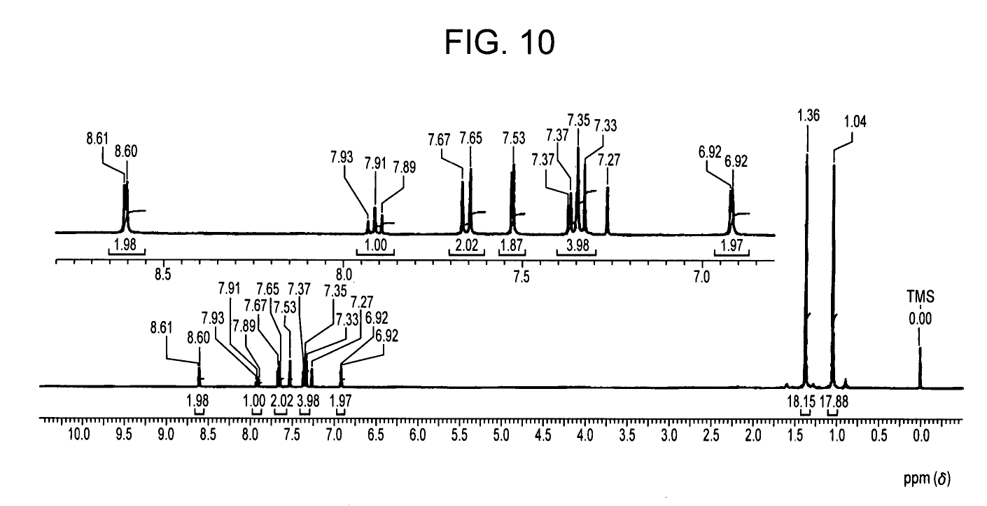

A compound represented by the foregoing structural formula (I-B-7) was obtained by following the same operations as in the foregoing Synthesis Example 1-B, except for changing the 4-chlorophenylhydrazine to 2,4-dichlorophenylhydrazine. Entire yield: 25.9%, MS: m/z 855 (M+). The IR spectrum and ¹H-NMR spectrum of this compound of the specific example (I-B-7) are shown in FIG. 9 and FIG. 10, respectively.

### Synthesis Example 3-B

### Synthesis of Compound of the Foregoing Specific Example (1-B-12)

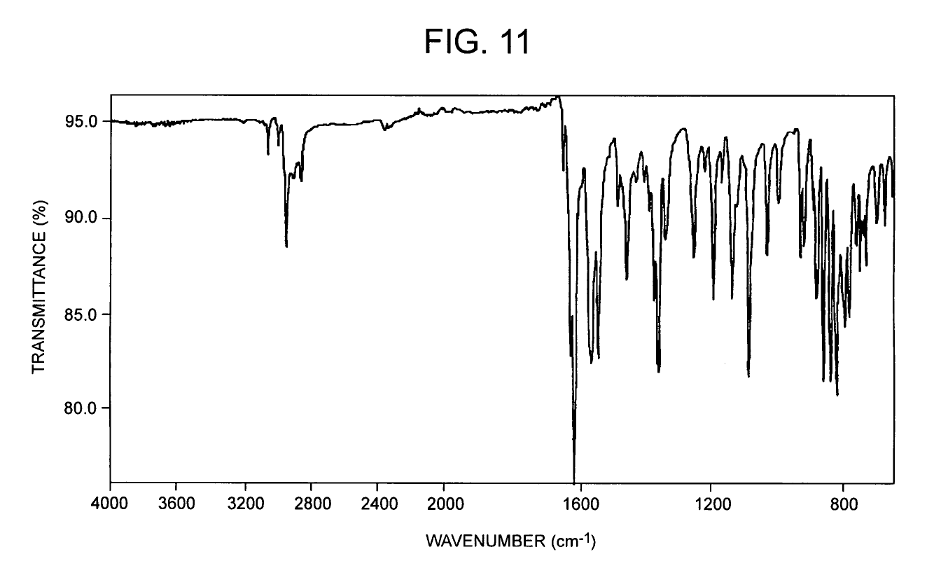

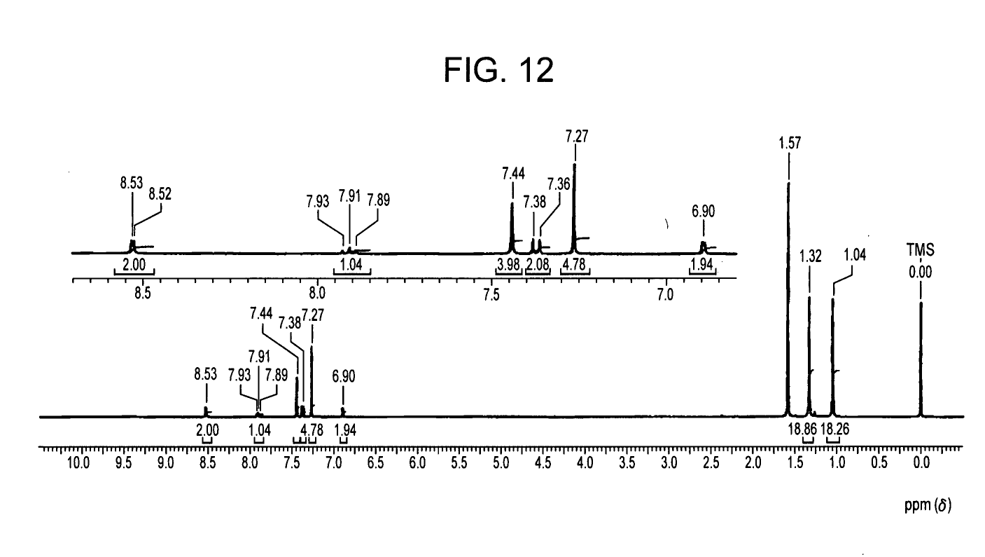

A compound represented by the foregoing structural formula (I-B-12) was obtained by following the same operations as in the foregoing Synthesis Example 1-B, except for changing the 4-chlorophenylhydrazine to 2,4,6-trichlorophenylhydrazine. Entire yield: 46.1%, MS: m/z 923 (M+). The IR spectrum and ¹H-NMR spectrum of this compound of the specific example (1-B-12) are shown in FIG. 11 and FIG. 12, respectively.

### Photoreceptor Example 1-B

A plate-like photoreceptor for evaluating electrical characteristics and a drum-like photoreceptor for evaluating printing were prepared, respectively.

A solution for undercoat layer as prepared in the following manner was coated on the external surface of each of an aluminum plate (3 cm×10 cm, thickness: 1 mm) and an aluminum drum (outer diameter: 30 mmφ, length: 247.5 mm, thickness: 0.75 mm) by the dip coating method and then dried at 100° C. for 60 minutes to remove the solvent. There was thus formed an undercoat layer having a film thickness of 0.3 μm.

(Preparation of Solution for Undercoat Layer)

B-a1) Soluble nylon (AMILAN CM8000, manufactured by Toray Industries, Inc.): 3 parts by weight (30 g)

The foregoing material B-a1) for the undercoat layer was stirred together with 97 parts by weight (970 g) of a mixed solvent of methanol/methylene chloride (5 vol./5 vol.) and dissolved to prepare a solution for the undercoat layer.

Next, on this undercoat layer, a dispersion for single layer type photosensitive layer as prepared in the following manner was coated by the dip coating method for the plate-like photoreceptor and by the ring coating method for the drum-like photoreceptor, respectively and then dried at 100° C. for 60 minutes to remove the solvent. Thus, a single layer type photosensitive layer having a film thickness of 25 μm was formed, from which was then prepared an electrophotographic photoreceptor.

(Preparation of Dispersion for Single Layer Type Photosensitive Layer)

B-b1) Charge generation substance: X-type metal-free phthalocyanine (see FIG. 2 of JP-A-2001-228637): 0.3 parts by weight (0.15 g)

B-b2) Hole transport substance: Triphenylamine compound represented by the following structural formula (HT1-101) ((HT1-101) of JP-A-2000-314969): 7 parts by weight (3.5 g)

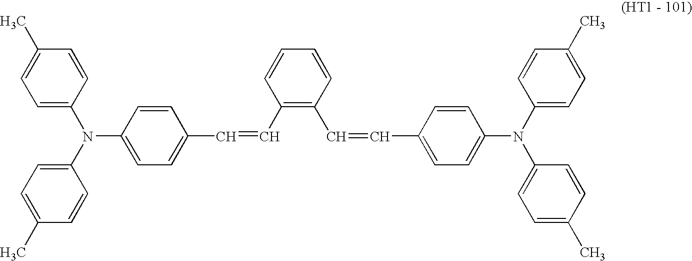

B-b3) Electron transport substance: Compound represented by the foregoing structural formula (1-B4): 2 parts by weight (1 g)

B-b4) Antioxidant: 3,5-Di-tert-4-hydroxytoluene (BHT) (manufactured by Tokyo Kasei Kogyo Co., Ltd.): 1 part by weight (0.5 g)

B-b5) Silicone oil: KF-50 (manufactured by Shin-Etsu Chemical Co., Ltd.): 0.01 parts by weight (0.005 g)

B-b6) Binder resin: Bisphenol Z type polycarbonate resin (PANLITE TS2020, manufactured by Teijin Chemicals Ltd.) ((BD1-1) of JP-A-2000-314969): 11 parts by weight (5.5 g)

In a 100-mL plastic bottle, the foregoing materials B-b1) to B-b6) for the undercoat photosensitive layer were charged together with 100 parts by weight (50 g) of a methylene chloride solvent and 50 g of stainless steel beads (3 mmφ), and the mixture was subjected to a dispersing treatment for 60 minutes by a paint conditioner Model 5400 (Red Devil Equipment Co., U.S.A.). Thereafter, the stainless steel beads were separated to prepare a dispersion for single layer type photosensitive layer.

### Photoreceptor Example 2-B

A single layer type photoreceptor was prepared in the same manner as in Photoreceptor Example 1-B, except that in the composition of the dispersion for photosensitive layer as used in Photoreceptor Example 1-B, 2 parts by weight of the compound represented by the foregoing structural formula (I-B4) as the electron transport substance was replaced by 2 parts by weight of the compound represented by the foregoing structural formula (I-B-7).

### Photoreceptor Example 3-B

A single layer type photoreceptor was prepared in the same manner as in Photoreceptor Example 1-B, except that in the composition of the dispersion for photosensitive layer as used in Photoreceptor Example 1-B, 2 parts by weight of the compound represented by the foregoing structural formula (I-B4) as the electron transport substance was replaced by 2 parts by weight of the compound represented by the foregoing structural formula (I-B-12).

### Photoreceptor Example 4-B

A single layer type photoreceptor was prepared in the same manner as in Photoreceptor Example 1-B, except that in the composition of the dispersion for photosensitive layer as used in Photoreceptor Example 1-B, 0.3 parts by weight of the X-type metal-free phthalocyanine as the charge generation substance was replaced by 0.2 parts by weight of Y-type titanyl phthalocyanine (see FIG. 4 of JP-A-2001-228637).

### Photoreceptor Example 5-B

A single layer type photoreceptor was prepared in the same manner as in Photoreceptor Example 1-B, except that in the composition of the dispersion for photosensitive layer as used in Photoreceptor Example 1-B, 7 parts by weight of the triphenylamine compound represented by the foregoing structural formula (HT1-101) as the hole transport substance was replaced by 7 parts by weight of a styryl compound represented by the following structural formula (HT2-2) ((HT2-2) of JP-A-2000-314969).

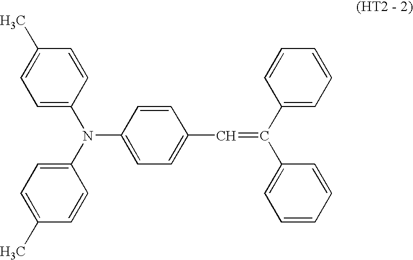

### Photoreceptor Example 6-B

A single layer type photoreceptor was prepared in the same manner as in Photoreceptor Example 1-B, except that in the composition of the dispersion for photosensitive layer as used in Photoreceptor Example 1-B, 7 parts by weight of the triphenylamine compound represented by the foregoing structural formula (HT1-101) as the hole transport substance was replaced by 7 parts by weight of a benzidine derivative represented by the following structural formula (HT-11) ((HT-11) of JP-A-2000-314969).

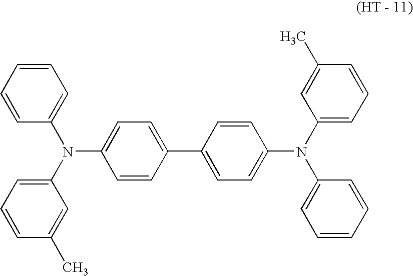

### Photoreceptor Example 7-B

A single layer type photoreceptor was prepared in the same manner as in Photoreceptor Example 1-B, except that in the composition of the dispersion for photosensitive layer as used in Photoreceptor Example 1-B, 0.3 parts by weight of the X-type metal-free phthalocyanine as the charge generation substance was replaced by 0.2 parts by weight of amorphous titanyl phthalocyanine (see FIG. 5 of JP-A-2001-228637).

### Photoreceptor Example 8-B

A single layer type photoreceptor was prepared in the same manner as in Photoreceptor Example 1-B, except that in the composition of the dispersion for photosensitive layer as used in Photoreceptor Example 1-B, 0.3 parts by weight of the X-type metal-free phthalocyanine as the charge generation substance was replaced by a bisazo pigment represented by the following formula (CG1-1).

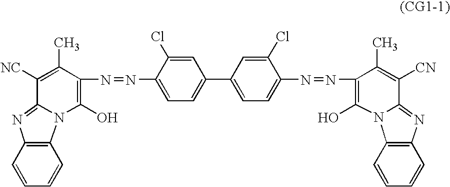

### Evaluation of Photoreceptor Examples 1-B to 8-B

Using a plate-like photoreceptor for evaluating electrical characteristics, the evaluation was carried out by an electrostatic copying paper analyzer EPA-8100 as manufactured by Kawaguchi Electric Works Co., Ltd.

First of all, the photoreceptor was charged in a dark place in an atmosphere at a temperature of 23° C. and a humidity of 45% such that the surface potential became about +700 V, and a retention rate Vₖ₅ of the surface potential after a lapse of 5 seconds was determined according to the following expression.
Retention rate Vₖ₅(%)=(V₅/V₀)×100

V₀: Surface potential immediately after charging

V₅: Surface potential 5 seconds after charging

Next, the surface potential was changed to +600 V, monochromic light of 1.0 μW/cm² resulting from separation of light of a halogen lamp into 780 nm (but into 550 nm with respect to the Photoreceptor Example 8-B) was exposed for 5 seconds, the amount of exposure required until the surface potential became one-half (+300 V) was determined as a sensitivity E_{1/2} (μj/cm²), and the surface potential 5 seconds after the exposure was determined as a residual potential Vᵣ (V).

The evaluation results are shown in the following Table 2.

TABLE 2

| | Retention | Sensitivity | Residual |
|---|---|---|---|
| | rate | E_{1/2} | potential |
| | Vₖ₅ (%) | (μJ/cm²) | Vᵣ (V) |
| Photoreceptor Example 1-B | 89.3 | 0.53 | 60 |
| Photoreceptor Example 2-B | 87.2 | 0.56 | 58 |
| Photoreceptor Example 3-B | 86.1 | 0.53 | 70 |
| Photoreceptor Example 4-B | 85.8 | 0.46 | 54 |
| Photoreceptor Example 5-B | 88.7 | 0.49 | 58 |
| Photoreceptor Example 6-B | 82.4 | 0.50 | 61 |
| Photoreceptor Example 7-B | 84.3 | 0.52 | 54 |
| Photoreceptor Example 8-B *⁾ | 79.7 | 0.61 | 69 |

*⁾ Light at 550 nm was employed for exposure only in Example 8-B.

Furthermore, a drum-like photoreceptor (30 mmφ) was used for evaluating the durability by actual printing. Each drum-like photoreceptor was installed in a laser printer HL-1240 as manufactured by Brother Industries, Ltd. and printed with a black solid image, a white solid image, and a half-tone image, respectively in an atmosphere at a temperature of 24° C. and a humidity of 53%. Subsequently, 5,000 sheets were printed with an image having a rate of printing (per page print coverage) of about 5%. Thereafter, a black solid image, a white solid image, and a half-tone image were again printed, respectively, thereby evaluating the image after printing 5,000 sheets.

As a result, in the photoreceptors of Photoreceptor Examples 1-B to 7-B, a good image was obtained in both the initial image and the image after printing 5,000 sheets.

Incidentally, with respect to Photoreceptor Example 8-B, the photoreceptor did not have sufficient sensitivity in a laser wavelength region (in the vicinity of 780 nm) of the used laser printer and therefore, it was noted that this photoreceptor is not suitable for this laser printer.

### Synthesis Example 1-C

### Synthesis of Compound of the Foregoing Specific Example (I-C-1)

A compound of the foregoing specific example (I-C-1) was synthesized according to the following reaction schemes (1-C-1) and (2-C-1).

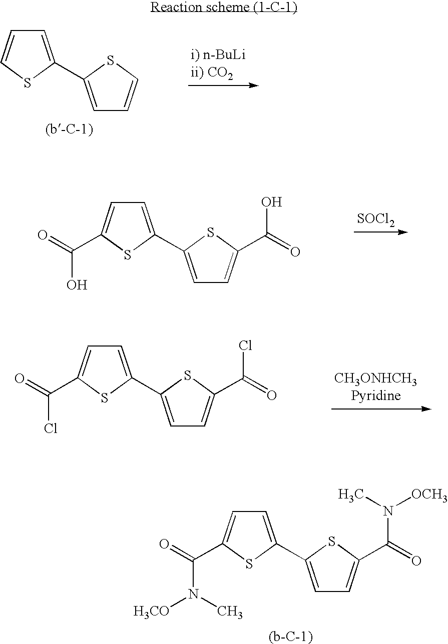

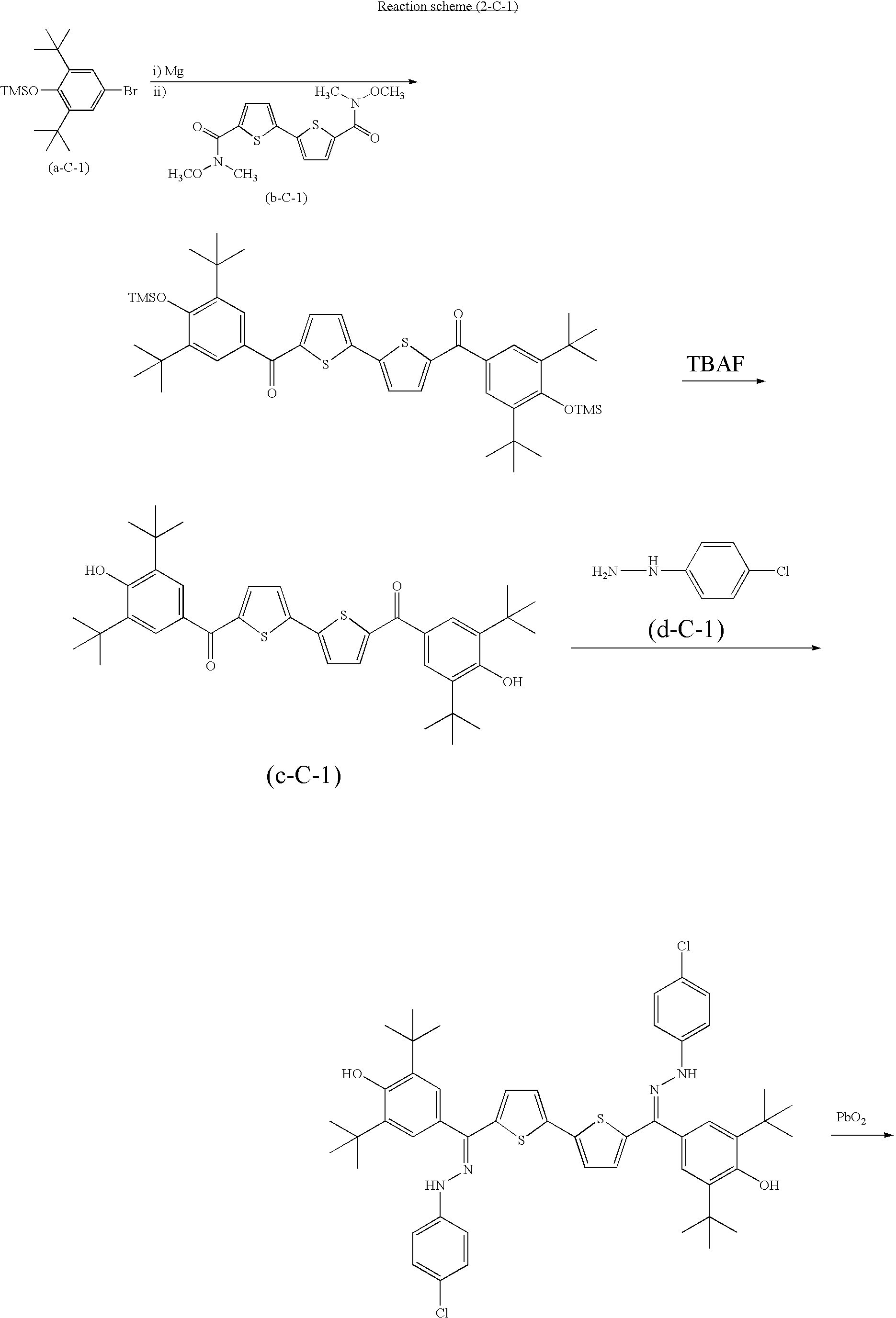

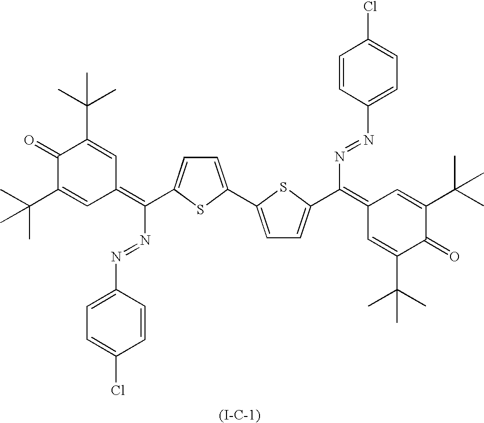

To a hexane solution of 100 mmoles (16.6 g) of bithiophene and 220 mmoles (25.6 g) of N,N,N,N-tetramethylethylenediamine, 220 mmoles (138 mL) of n-BuLi was added dropwise in an ice bath in a nitrogen atmosphere. Thereafter, the mixture was agitated at room temperature for 0.5 hours and additionally refluxed under heat for 0.5 hours. The reaction solution was cooled and then poured into dry ice, followed by allowing it to stand overnight. The resulting reaction solution was extracted with chloroform and a 10% sodium hydroxide aqueous solution. When the resulting aqueous solution was made acidic with hydrochloric acid, a precipitate was deposited. By performing filtration, bithiophenedicarboxylic acid was obtained as a crude product in a yield of 19.3 g (percent yield: 75.9%).

To 50 mmoles (12.7 g) of the resulting bithiophenedicarboxylic acid, 150 mmoles (17.8 g) of thionyl chloride was added, and three drops of N,N-dimethylformamide was added dropwise, followed by refluxing under heat. After a lapse of 2 hours, the excessive thionyl chloride was evaporated off to obtain a crude product. To a dichloromethane solution of this compound and 110 mmoles (10.7 g) of N,O-dimethylhydroxyamine hydrochloride, 330 mmoles (26.1 g) of pyridine was added in a nitrogen atmosphere at room temperature, and the mixture was stirred for 2 hours. Thereafter, the reaction solution was poured into aqueous hydrochloric acid and extracted with dichloromethane, followed by concentration. There was thus obtained, as a crude product, a compound represented by the foregoing structural formula (b-C-1) in a yield of 8.2 g (48.2%).

Next, to a THF solution of 10 mmoles (3.4 g) of the compound represented by the foregoing structural formula (b-C-1), a Grignard reagent as prepared from 36 mmoles (0.9 g) of magnesium and a THF solution of 30 mmoles (10.7 g) of 4-bromo-2,6-di-t-butyl-1-(trimethylsiloxy)benzene (the foregoing structural formula (a-C-1)) was added dropwise, and the mixture was stirred at room temperature for 3 hours. Thereafter, a small amount of a 1N hydrochloric acid aqueous solution was added to stop the reaction. In addition, 30 mmoles (30 mL) of a THF solution of 1.0 M tetrabutylammonium fluoride (TBAF) was added, followed by stirring. The reaction solution was poured into aqueous hydrochloric acid and extracted with dichloromethane, followed by concentration. There was thus obtained, as a crude product, a compound represented by the foregoing structural formula (c-C-1) in a yield of 2.6 g (41.7%)

In addition, 4 mmoles (2.5 g) of the compound represented by the foregoing structural formula (c-C-1) and 40 mmoles (7.2 g) of 4-chlorophenylhydrazine hydrochloride (the foregoing structural formula (d-C-1)) were dissolved in pyridine, and the mixture was refluxed under heat. The reaction solution was poured into aqueous hydrochloric acid and extracted with dichloromethane, followed by concentration. Thereafter, the resulting product was purified by column chromatography to obtain a crude product.

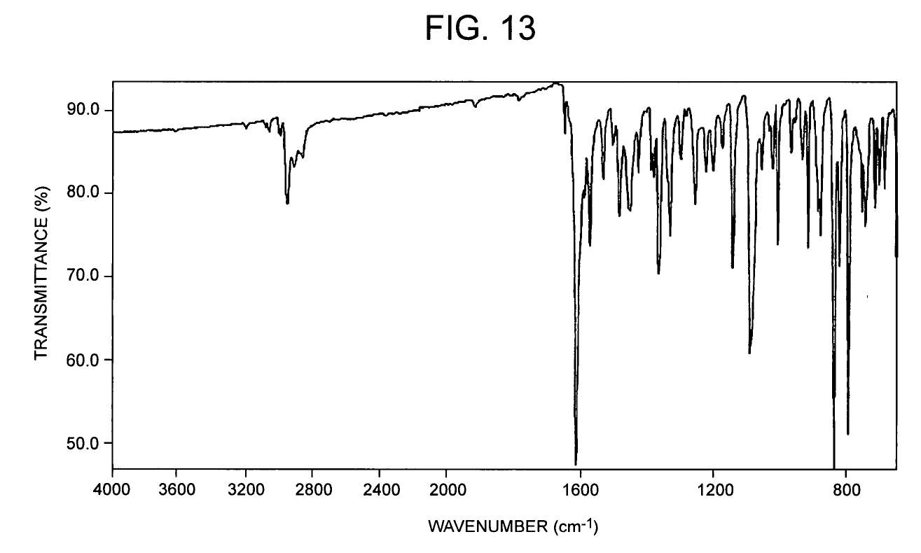

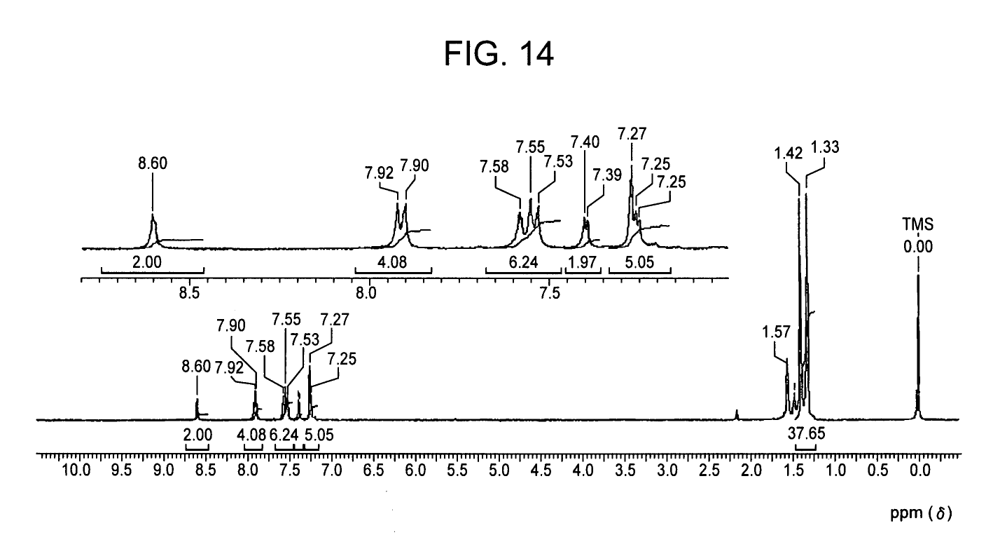

To a chloroform solution of the foregoing crude product, 6.9 mmoles (1.7 g) of lead dioxide (PbO₂) was added at room temperature, and the mixture was stirred. After filtering out the residue, the reaction solution was concentrated, and the resulting solid was recrystallized from a mixed solution of chloroform and hexane to obtain a compound represented by the foregoing structural formula (I-C-1). Yield: 2.0 g (percent yield: 57.1%), MS: m/z 874 (M+). Incidentally, the entire yield was 8.7%. The IR spectrum and ¹H-NMR spectrum of this compound of the structural formula (1-C-1) are shown in FIG. 13 and FIG. 14, respectively.

Incidentally, the 4-bromo-2,6-di-tert-butyl-1-(trimethylsiloxy)benzene (the foregoing structural formula (a-C-1)) as used in the foregoing Synthesis Example can be, for example, synthesized by a known method as described in JP-A-2001-222122 and so on.

### Photoreceptor Example 1-C

20 parts by weight (2 g) of X-type metal-free phthalocyanine (H₂Pc) and 100 parts by weight (10 g) of the compound represented by the foregoing structural formula (I-C-1) were kneaded together with 100 parts by weight (10 g) of a polyester resin (trade name: VYLON 200, manufactured by Toyobo Co., Ltd.) and a tetrahydrofuran (THF) solvent for 3 hours by using a mixer to prepare a coating liquid. The coating liquid was coated on an aluminum-made drum having an outer diameter of 30 mm and a length of 260 mm as an electrically conductive substrate to form a photosensitive layer in a film thickness after drying of 15 μm, from which was then prepared a photoreceptor.

### Photoreceptor Example 2-C

A single layer type photoreceptor was prepared in the same manner as in Photoreceptor Example 1-C, except that in Photoreceptor Example 1-C, the compound represented by the foregoing structural formula (1-C-3) was used in place of the compound represented by the foregoing structural formula (1-C-1).

### Photoreceptor Example 3-C

A single layer type photoreceptor was prepared in the same manner as in Photoreceptor Example 1-C, except that in Photoreceptor Example 1-C, the compound represented by the foregoing structural formula (I-C-5) was used in place of the compound represented by the foregoing structural formula (I-C-1).

### Photoreceptor Example 4-C

2 parts by weight (0.2 g) of X-type metal-free phthalocyanine (H₂Pc), 40 parts by weight (4 g) of the compound represented by the structural formula (I-C-7), 60 parts by weight (6 g) of a benzidine derivative represented by the following structural formula (HT-11) as a hole transport substance, and 100 parts by weight (10 g) of a polycarbonate resin (trade name: PCZ-200, manufactured by Mitsubishi Gas Chemical Company, Inc.) were kneaded together with methylene chloride for 3 hours by using a mixer to prepare a coating liquid. The coating liquid was coated on the same aluminum-made drum as in Example 1-C to form a photosensitive layer in a film thickness after drying of 25 μm, from which was then prepared a single layer type photoreceptor.

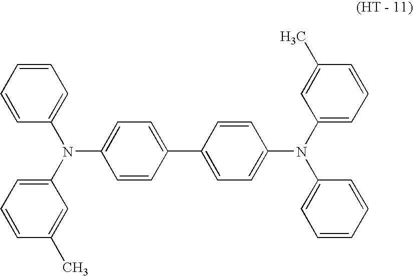

### Photoreceptor Example 5-C

A single layer type photoreceptor was prepared in the same manner as in Photoreceptor Example 4-C, except that in Photoreceptor Example 4-C, a squarylium compound represented by the following structural formula was used in place of the X-type metal-free phthalocyanine and that the compound represented by the foregoing structural formula (I-C-11) was used in place of the compound represented by the foregoing structural formula (I-C-7).

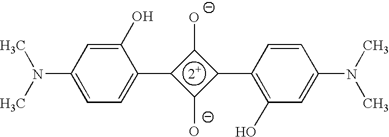

### Photoreceptor Example 6-C

70 parts by weight (7 g) of α-type titanyl phthalocyanine (TiOPc) and 30 parts by weight (3 g) of a vinyl chloride copolymer (trade name: MR-110, manufactured by Zeon Corporation) were kneaded together with methylene chloride for 3 hours by using a mixer to prepare a coating liquid. The coating liquid was coated on the same aluminum-made drum as in Example 1-C to form a charge generation layer in a film thickness after drying of 1 μm. Next, 100 parts by weight (10 g) of the compound represented by the foregoing structural formula (I-C-1), 100 parts by weight (10 g) of a polycarbonate resin (trade name: PCZ-200, manufactured by Mitsubishi Gas Chemical Company, Inc.), and 0.1 parts by weight (0.01 g) of silicone oil were mixed with methylene chloride. The mixture was coated on the charge generation layer to form a charge transport layer in a film thickness after drying of 7 μm. There was thus prepared a layered type photoreceptor.

### Photoreceptor Example 7-C

A single layer type photoreceptor was prepared in the same manner as in Photoreceptor Example 4-C, except that in Photoreceptor Example 4-C, a bisazo pigment represented by the following structural formula (CG1-1) was used in place of the X-type metal-free phthalocyanine and that the compound represented by the foregoing structural formula (I-C-6) was used in place of the compound represented by the foregoing structural formula (I-C-7).

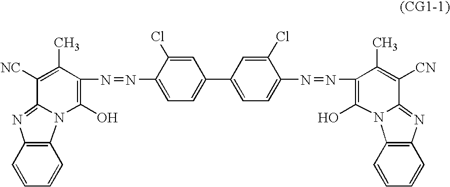

### Photoreceptor Example 8-C

A single layer type photoreceptor was prepared in the same manner as in Photoreceptor Example 4-C, except that in Photoreceptor Example 4-C, a bisazo pigment represented by the following structural formula was used in place of the X-type metal-free phthalocyanine and that the compound represented by the foregoing structural formula (I-C-12) was used in place of the compound represented by the foregoing structural formula (I-C-7).

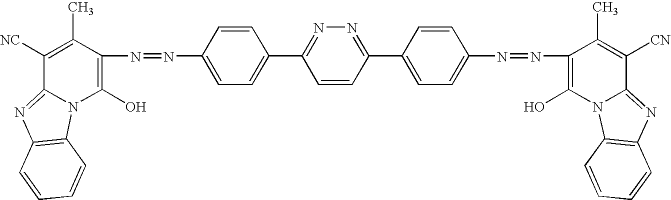

Electrophotographic characteristics of each of the thus obtained photoreceptors of the Examples were measured.

An initial surface potential when the surface of the photoreceptor was positively charged by performing corona discharge of +4.5 kV in a dark place was designated as Vₛ (V), and a surface potential V_{d} (V) when the photoreceptor was subsequently held in a dark place for 5 seconds in the state that the corona discharge was stopped was measured. In addition, the surface of the photoreceptor was subsequently irradiated with white light at an illuminance of 100 lux, and a time (seconds) required until the V_{d} became one-half was determined and defined as sensitivity E_{1/2} (lux.s). Furthermore, a surface potential when the photoreceptor was irradiated with white light at an illuminance of 100 lux was designated as a residual potential Vᵣ (V). Moreover, with respect to the photoreceptors of Photoreceptor Examples 1-C to 6-C, since high sensitivity by long wavelength light can be expected, electrophotographic characteristics when monochromic light having a wavelength of 780 nm was used were measured at the same time. That is, the measurement was carried out in the same manner till V_{d}. Monochromic light (780 nm) of 1 μW was then irradiated in place of the white light, and a time (seconds) required until the V_{d} became one-half was determined to obtain sensitivity E_{1/2} (μJ/cm²). Also, a residual potential Vᵣ (V) when this light was irradiated on the surface of the photoreceptor for 10 seconds was measured. The results of these evaluations are shown in Table 3.

TABLE 3

| | White light | | Monochromic light of 780 nm | |
|---|---|---|---|---|
| | | Residual | | Residual |
| | Sensitivity | potential | Sensitivity | potential |
| | (lux.s) | (V) | (μJ/cm²) | (V) |
| Photoreceptor | 1.2 | 100 | 0.9 | 80 |
| Example 1-C | | | | |
| Photoreceptor | 1.0 | 80 | 0.7 | 70 |
| Example 2-C | | | | |
| Photoreceptor | 0.7 | 80 | 0.7 | 50 |
| Example 3-C | | | | |
| Photoreceptor | 0.9 | 70 | 0.8 | 50 |
| Example 4-C | | | | |
| Photoreceptor | 0.8 | 70 | 0.6 | 40 |
| Example 5-C | | | | |
| Photoreceptor | 1.8 | 140 | 1.8 | 130 |
| Example 6-C | | | | |
| Photoreceptor | 1.2 | 90 | — | — |
| Example 7-C | | | | |
| Photoreceptor | 1.1 | 90 | — | — |
| Example 8-C | | | | |

1. A compound having a structure represented by the following general formula (I): 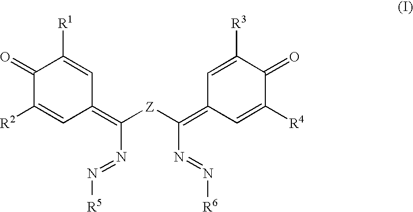
wherein in the general formula (I), R¹, R², R³, and R⁴ can be the same or different and each is a hydrogen atom, an optionally substituted alkyl group having from 1 to 12 carbon atoms, or an optionally substituted aryl group; R⁵ and R⁶ can be the same or different and each is an optionally substituted aryl group or an optionally substituted heterocyclic group; and Z represents a structure having the following general formula (F-A), (F-B) or (F-C): 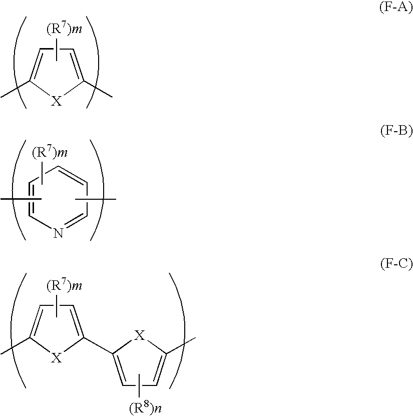
wherein in the formulae, R⁷ and R⁸ can be the same or different and each is an optionally substituted alkyl group having from 1 to 12 carbon atoms; m and n each represents an integer of from 0 to 2; X is a sulfur atom or an oxygen atom; and any substituent of any of R¹ to R⁸ is a halogen atom, a nitro group, an alkyl group, an aryl group, a heterocyclic group, a halogenated alkyl group, or an alkoxy group, and any two adjacent substituents of any of R¹ to R⁸ can be taken together to form a ring.

2. The novel compound according to claim 1, having a structure represented by the following general formula (I-A): 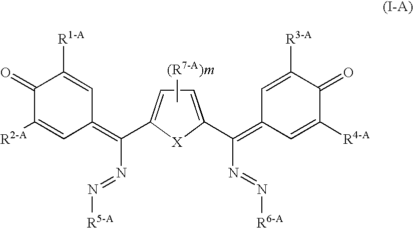
wherein in the formula (I-A), R^{1-A}, R^{2-A}, R^{3-A}, and R^{4-A} can be the same or different and each is a hydrogen atom, an optionally substituted alkyl group having from 1 to 12 carbon atoms, or an optionally substituted aryl group; R^{5-A} and R^{6-A} can be-the same or different and each is an optionally substituted aryl group or an optionally substituted heterocyclic group; R^{7-A} is an optionally substituted alkyl group having from 1 to 12 carbon atoms; X is a sulfur atom or an oxygen atom; m represents an integer of from 0 to 2; and any substituent of any of R^{1-A} to R^{7-A} is a halogen atom, a nitro group, an alkyl group having from 1 to 6 carbon atoms, an aryl group, a halogenated alkyl group having from 1 to 6 carbon atoms, or an alkoxy group having from 1 to 6 carbon atoms.

3. The compound according to claim 1, having a structure represented by the following general formula (I-B): 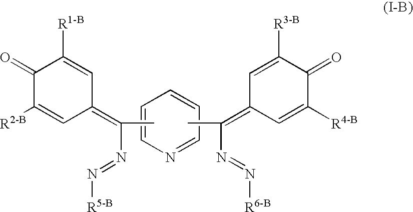
wherein in the formula (I-B), R^{1-B}, R^{2-B}, R^{3-B}, and R^{4-B} can be the same or different and each is a hydrogen atom or an optionally substituted alkyl group having from 1 to 12 carbon atoms; R^{5-B} and R^{6-B} can be the same or different and each is an optionally substituted aryl group or an optionally substituted heterocyclic group; and any substituent of any of R^{1-B} to R^{6-B} is a halogen atom, an alkyl group, an alkoxy group, an aryl group, a heterocyclic group, a fluorinated alkyl group, or a nitro group, and any two adjacent substituents of any of R^{1-B} to R^{6-B} can be-taken together to form a ring.

4. The compound according to claim 1, having a structure represented by the following general formula (I-C): 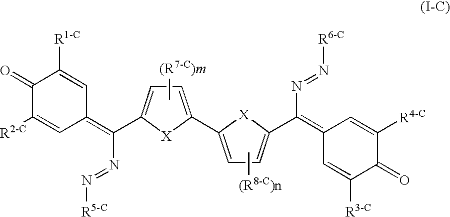
wherein in the formula (I-C), R^{1-C}, R^{2-C}, R^{3-C}, and R^{4-C} can be the same or different and each is a hydrogen atom, an optionally substituted alkyl group having from 1 to 6 carbon atoms, or an optionally substituted aryl group; R^{5-C} and R^{6-C} can be the same or different and each is an optionally substituted aryl group or a heterocyclic group; R^{7-C} and R^{8-C} each is a hydrogen atom or an optionally substituted alkyl group having from 1 to 10 carbon atoms; X is a sulfur atom or an oxygen atom; m and n each represents an integer of from 1 to 2; and any substituent of any of R^{1-C} to R^{8-C} is a halogen atom, a nitro group, an alkyl group, an aryl group, a heterocyclic group, a halogenated alkyl group, or an alkoxy group, and any two adjacent substituents of any of R^{1-C} to R^{8-C} can be taken together to form a ring.

5. An electrophotographic photoreceptor including an electrically conductive substrate having thereon a photosensitive layer containing a charge generation substance and a charge transport substance that includes at least one compound having electron transport properties as represented by the following general formula (I): 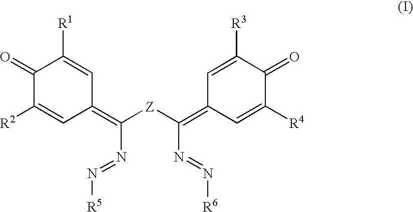
wherein in the general formula (I), R¹, R², R³, and R⁴ can be the same or different and each is a hydrogen atom, an optionally substituted alkyl group having from 1 to 12 carbon atoms, or an optionally substituted aryl group; R⁵ and R⁶ can be the same or different and each is an optionally substituted aryl group or an optionally substituted heterocyclic group; and Z represents a structure having the following general formula (F-A), (F-B) or (F-C): 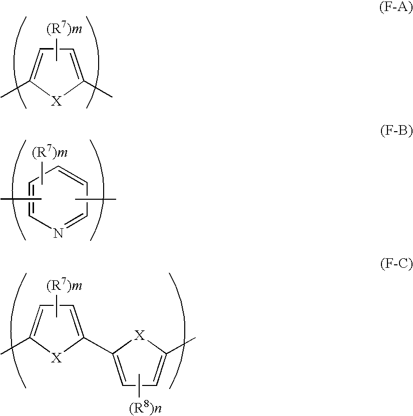
wherein in the formulae, R⁷ and R⁸ can be the same or different and each is an optionally substituted alkyl group having from 1 to 12 carbon atoms; m and n each represents an integer of from 0 to 2; X is a sulfur atom or an oxygen atom; and any substituents of any of R¹ to R⁸ is a halogen atom, a nitro group, an alkyl group, an aryl group, a heterocyclic group, a halogenated alkyl group, or an alkoxy group, and any two adjacent substituents of any of R¹ to R⁸ can be taken together to form a ring.

6. The electrophotographic photoreceptor including an electrically conductive substrate having thereon a photosensitive layer containing a charge generation substance and a charge transport substance according to claim 5, containing, as said charge transport substance, at least one compound having electron transport properties as represented by the following general formula (I-A): 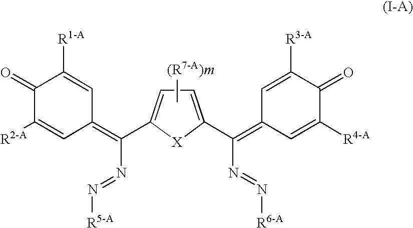
wherein in the formula (I-A), R^{1-A}, R^{2-A}, R^{3-A}, and R^{4-A} can be the same or different and each is a hydrogen atom, an optionally substituted alkyl group having from 1 to 12 carbon atoms, or an optionally substituted aryl group; R^{5-A} and R^{6-A} can be the same or different and each is an optionally substituted aryl group or an optionally substituted heterocyclic group; R^{7-A} is an optionally substituted alkyl group having from 1 to 12 carbon atoms; X is a sulfur atom or an oxygen atom; m represents an integer of from 0 to 2; and any substituents of any of R^{1-A} to R^{7-A} is a halogen atom, a nitro group, an alkyl group having from 1 to 6 carbon atoms, an aryl group, a halogenated alkyl group having from 1 to 6 carbon atoms, or an alkoxy group having from 1 to 6 carbon atoms.

7. The electrophotographic photoreceptor including an electrically conductive substrate having thereon a photosensitive layer containing a charge generation substance and a charge transport substance according to claim 5, containing, as said charge transport substance, at least one compound having electron transport properties as represented by the following general formula (I-B): 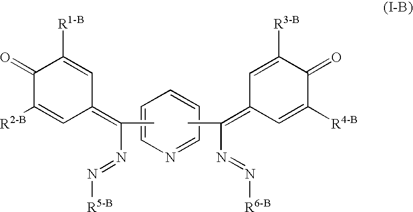
wherein in the formula (I-B), R^{1-B}, R^{2-B}, R^{3-B}, and R^{4-B} can be the same or different and each is a hydrogen atom or an optionally substituted alkyl group having from 1 to 12 carbon atoms; R^{5-B} and R^{6-B} can be the same or different and each is an optionally substituted aryl group or an optionally substituted heterocyclic group; and any substituents of any of R^{1-B} to R^{6-B} is a halogen atom, an alkyl group, an alkoxy group, an aryl group, a heterocyclic group, a fluorinated alkyl group, or a nitro group, and any substituent of any of R^{1-B} to R^{6-B} can be taken together to form a ring.

8. The electrophotographic photoreceptor including an electrically conductive substrate having thereon a photosensitive layer containing a charge generation substance and a charge transport substance according to claim 5, containing, as said charge transport substance, at least one compound having electron transport properties as represented by the following general formula (I-C): 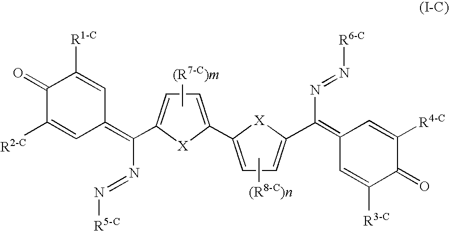
wherein in the formula (I-C), R^{1-C}, R^{2-C}, R^{3-C}, and R^{4-C} can be the same or different and each is a hydrogen atom, an optionally substituted alkyl group having from 1 to 6 carbon atoms, or an optionally substituted aryl group; R^{5-C} and R^{6-C} can be the same or different and each is an optionally substituted aryl group or a heterocyclic group; R^{7-C} and R^{8-C} each is a hydrogen atom or an optionally substituted alkyl group having from 1 to 10 carbon atoms; X is a sulfur atom or an oxygen atom; m and n each represents an integer of from 1 to 2; and any substituent of any of R^{1-C} to R^{8-C} is a halogen atom, a nitro group, an alkyl group, an aryl group, a heterocyclic group, a halogenated alkyl group, or an alkoxy group, and any substituents of any of R^{1-C} to R^{8-C} can be taken together to form a ring.

9. An electrophotographic photoreceptor including an electrically conductive substrate having thereon directly or via the undercoat layer a single layer type photosensitive layer containing a charge generation substance, a charge transport substance, and a resin binder, containing, as said charge transport substance, a hole transport substance and at least one compound having electron transport properties as represented by the following general formula (I): 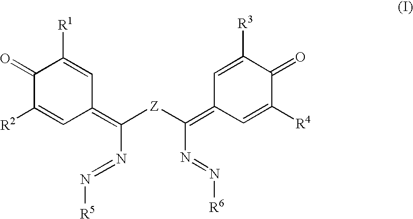
wherein in the general formula (I), R¹, R², R³, and R⁴ can be the same or different and each is a hydrogen atom, an optionally substituted alkyl group having from 1 to 12 carbon atoms, or an optionally substituted aryl group; R⁵ and R⁶ can be the same or different and each is an optionally substituted aryl group or an optionally substituted heterocyclic group; and Z represents a structure having the following general formula (F-A), (F-B) or (F-C): 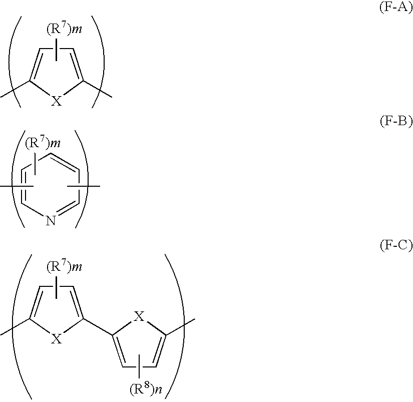
wherein in the formulae, R⁷ and R⁸ can be the same or different and each is an optionally substituted alkyl group having from 1 to 12 carbon atoms; m and n each represents an integer of from 0 to 2; X is a sulfur atom or an oxygen atom; and any substituent of any of R¹ to R⁸ is a halogen atom, a nitro group, an alkyl group, an aryl group, a heterocyclic group, a halogenated alkyl group, or an alkoxy group, and any two adjacent substituents of any of R¹ to R⁸ can be taken together to form a ring.

10. The electrophotographic photoreceptor including an electrically conductive substrate having thereon directly or via the undercoat layer a single layer type photosensitive layer containing a charge generation substance, a charge transport substance, and a resin binder according to claim 9, containing, as said charge transport substance, a hole transport substance and at least one kind of a compound having electron transport properties as represented by the following general formula (I-A): 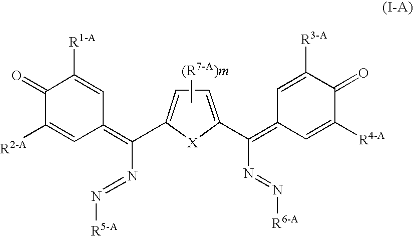
wherein in the formula (I-A), R^{1-A}, R^{2-A}, R^{3-A}, and R^{4-A} can be the same or different and each is a hydrogen atom, an optionally substituted alkyl group having from 1 to 12 carbon atoms, or an optionally substituted aryl group; R^{5-A} and R^{6-A} can be the same or different and each is an optionally substituted aryl group or an optionally substituted heterocyclic group; R^{7-A} is an optionally substituted alkyl group having from 1 to 12 carbon atoms; X is a sulfur atom or an oxygen atom; m represents an integer of from 0 to 2; and any substituent of any of R^{1-A} to R^{7-A} is a halogen atom, a nitro group, an alkyl group having from 1 to 6 carbon atoms, an aryl group, a halogenated alkyl group having from 1 to 6 carbon atoms, or an alkoxy group having from 1 to 6 carbon atoms.

11. The electrophotographic photoreceptor including an electrically conductive substrate having thereon directly or via the undercoat layer a single layer type photosensitive layer containing a charge generation substance, a charge transport substance, and a resin binder according to claim 9, containing, as said charge transport substance, a hole transport substance and at least one kind of a compound having electron transport properties as represented by the following general formula (I-B): 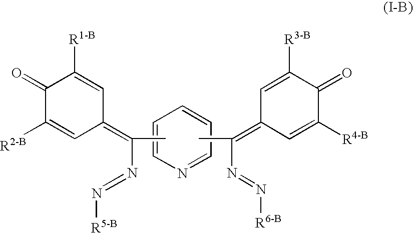
wherein in the formula (I-B), R^{1-B}, R^{2-B}, R^{3-B}, and R^{4-B} can be the same or different and each is a hydrogen atom or an optionally substituted alkyl group having from 1 to 12 carbon atoms; R^{5-B} and R^{6-B} can be the same or different and each is an optionally substituted aryl group or an optionally substituted heterocyclic group; and any substituent of any of R^{1-B} to R^{6-B} is a halogen atom, an alkyl group, an alkoxy group, an aryl group, a heterocyclic group, a fluorinated alkyl group, or a nitro group, and any two adjacent substituents of any of R^{1-A} to R^{6-B} can be taken together to form a ring.

12. The electrophotographic photoreceptor including an electrically conductive substrate having thereon directly or via the undercoat layer a single layer type photosensitive layer containing a charge generation substance, a charge transport substance, and a resin binder according to claim 9, containing, as said charge transport substance, a hole transport substance and at least one kind of a compound having electron transport properties as represented by the following general formula (I-C): 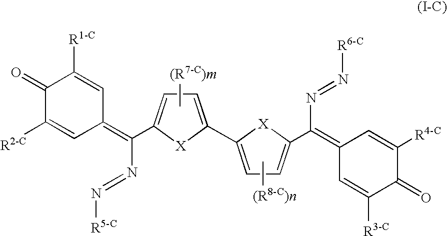
wherein in the is a hydrogen atom, an optionally substituted alkyl group having from 1 to 6 carbon atoms, or an optionally substituted aryl group; R^{5-C} and R^{6-C} can be the same or different and each is an optionally substituted aryl group or a heterocyclic group; R^{7-C} and R^{8-C} each is a hydrogen atom or an optionally substituted alkyl group having from 1 to 10 carbon atoms; X is a sulfur atom or an oxygen atom; m and n each represents an integer of from 1 to 2; and any substituent of any of R^{1-C} to R^{8-C} is a halogen atom, a nitro group, an alkyl group, an aryl group, a heterocyclic group, a halogenated alkyl group, or an alkoxy group, and any two adjacent substituents of any of R¹ to R⁸ can be taken together to form a ring.

13. An electrophotographic apparatus which is characterized by being provided with the electrophotographic photoreceptor according to claim 5 and performing a charge process by a positive charge process.
